SEQUENCE LISTING (1 sequence)

SEQ ID NO: 1 - DNA, 1041 bp, Mus musculus
atggcgctgctcaaagtcaagtttgaccagaagaagcgggtcaagttggcccaggggctctggcttatgaactggctgtccgtgttggccggcatcgtcctcttcagcttggggctgttcttgaagattgaacttcgcaagaggagcgaagtgatgaataattctgagagccactttgtgcccaactccctgataggggtgggggtcctgtcctgtgtcttcaactctctggctgggaagatctgctatgatgccctggacccggccaagtacgccaagtggaagccctggctgaagccgtacctggctgtctgcatcttctttaacgtcatcctcttcctggtggctctctgctgctttctgttgcggggctccctggagagcaccctggcttacggactcaagaatgggatgaagtattatcgggatacggacacccccggccggtgcttcatgaaaaagaccatcgacatgctccagattgagttcaagtgctgtgggaacaacggcttccgggactggttcgagattcagtggatcagcaatcgctacctggacttctcctccaaggaggtcaaagatcgcatcaagagcaacgtggatgggcggtacctggtggacggcgtccctttcagctgctgcaaccccagctccccgcggccctgtatccagtaccagctcaccaacaactcggcgcactacagctatgaccatcagactgaggagctcaacctctggctgcggggctgcagggccgctctgctgaattactacagcagcctcatgaattccatgggcgtcgtcacacttctcgtctggctctttgaggtgagcatcactgccggactccgctacctccacacagcgctggagagtgtgtctaacccggaggaccccgagtgtgagagtgagggctggctgctggagaagagcgtgcccgagacctggaaggcctttctggagagctttaagaagctgggcaagagcaatcaggtggaggctgaaggtgcagacgcaggccaagcgcctgaagccggctga

(19) United States
(12) Patent Application Publication
(10) Pub. No.: US 2009/0011040 A1
(43) Pub. Date: Jan. 8, 2009

(54) Use of compacted nucleic acid nanoparticles in non-viral treatments of ocular diseases

(76) Inventors: Muna I. Naash, Oklahoma City, OK (US); Mark J. Cooper, Moreland Hills, OH (US)

Correspondence Address: DUNLAP CODDING, P.C., PO BOX 16370, OKLAHOMA CITY, OK, 73113, US

(21) Appl. No.: 12/150,986

(22) Filed: May 2, 2008

Related U.S. Application Data
(60) Provisional application No. 60/927,388, filed on May 2, 2007.

(51) Int. Cl. A61K 9/14; A61K 31/7052; A61P 27/02; A61K 48/00

(52) U.S. Cl. 424/501; 514/44; 977/773; 977/906

(57) ABSTRACT

The present invention is a method of using compacted nucleic acid (such as DNA) nanoparticles for non-viral gene transfer to various tissues of the human eye or eyes of other mammals. These nanoparticles comprise, in one embodiment, a neutrally-charged complex containing a single molecule of plasmid DNA compacted with polyethylene glycol (PEG)-substituted poly lysine peptides.

### CROSS-REFERENCE TO RELATED APPLICATIONS

This present application claims the benefit under 35 U.S.C. 119(e) of U.S. Provisional Application Ser. No. 60/927,388, filed May 2, 2007, the entirety of which is hereby expressly incorporated herein by reference.

### STATEMENT REGARDING FEDERALLY SPONSORED RESEARCH OR DEVELOPMENT

Some aspects of this invention were made in the course of Grants EY016201, EY10609, EY007361, EY018656, and Core Grant for Vision Research EY12190 awarded by the National Institutes of Health, therefore the Government has rights in some aspects of this invention.

### BACKGROUND

The eye is comprised of several specialized tissues that work together to initiate visual perception in response to photons of light. Any insult to these tissues results in a consequence to vision and an impact on the quality of life for the patient. Both environmental trauma and genetic disorders can cause varying degrees of ocular diseases. Current therapies for ocular disorders are often surgically-based or topical treatments however they often fail to correct the underlying genetic deficit. As the eye is easily accessible and immune-privileged, the use of gene transfer is an attractive therapeutic option for numerous forms of blinding disorders.

Many disease-causing mutations and their contribution to the pathogenesis of ocular diseases including cataracts, glaucoma, and retinitis pigmentosa have been well characterized [1-5]. In addition, several treatment strategies for overcoming these genetic deficits have been attempted and proven in tissue culture and various animal models [6-10]. Many attempts to rescue genetic deficits using viral vectors for gene therapy have proven effective in the eye [11-15]. Encouraging results have been shown for example in Briard dogs harboring a naturally occurring mutation in the RPE65 gene, which causes visual impairment similar to Early Onset Severe Retinal Dystrophy in humans [16, 17]. Bennett and colleagues used adeno-associated virus to express the RPE65 cDNA which restored retinal function and has successfully persisted over 3 years. Although viral vectors also have been successful in alleviating hereditary retinal degeneration in mice [12, 18], they can be limited by cell tropism, the size of the expression cassette to be transferred, and host immunity to repeat infections [19, 20]. For example, viral vectors are immunogenic and repeat applications have often resulted in production of neutralizing antibodies by the host, preventing productive readministration. Inflammatory responses to some vectors, such as adenovirus, have also resulted in toxic responses, including mortality. The use of viruses as vehicles to deliver exogenous genes has also demonstrated insertional mutagenesis due to chromosome integration. Additionally, concerns regarding the safety of using viral vectors in human patients have been raised and some trials have resulted in oncogenesis or even mortality [19, 21-23].

Therefore, other methods of gene therapy which do not rely on viral vectors would be desirable and consequently comprise an objective of the present invention.

### BRIEF DESCRIPTION OF THE DRAWINGS

FIG. 1 is an electron micrograph showing shapes of nanoparticles formed using trifluoroacetate (TFA) and acetate (AC) as counterions. During compaction of the gene expression plasmids, the presence of TFA as a polylysine peptide counterion produces ellipsoidal nanoparticles with a minor diameter of about 22 nm or less whereas acetate as the polylysine peptide counterion produces rods with a minor diameter of about 8 nm. Scale bar, 100 nM.

FIG. 2 shows graphs indicating that injection of NMP (SEQ ID NO:1) nanoparticles into P5 rds^{+/-} animals increases Rds mRNA levels. cDNA from eyes injected with saline, naked plasmid DNA (a) or nanoparticle DNA (b) at PI-2 through PI-120 was collected and analyzed by qRT-PCR to determine total Rds mRNA levels relative to HPRT. Because Rds primers amplify both native (endogenous) and transferred (exogenous) Rds, expression values are reported as fold change from the uninjected contralateral control eye. Values shown are averages ± S. D. (N=3-4 mice per group). (a) Injection of saline or naked NMP plasmid DNA does not alter Rds message levels at any time point. (b) Conversely, injection of both CBA-NMP and IRBP-NMP compacted DNA nanoparticles leads to a significant, two- to four-fold increase in total Rds message level compared to the uninjected control eye. This increase persists through the last time point examined (PI-120).

FIG. 3. Distribution of transferred Rds (NMP) is similar to that of normal (endogenous) Rds. Frozen retinal sections from eyes collected at multiple ages (PI-2 to PI-30) were immunostained for NMP (3B6, green) and total Rds (Rds-CT, red) with a nuclear counterstain (DAPI, blue). Transferred Rds from eyes injected with CBA-NMP (a) and IRBP-NMP (b) nanoparticles is detected beginning at PI-2. Expression remains strong through the latest time point analyzed (PI-30) and co-localizes with native Rds. Expression is limited to the OSs or nascent OSs and is not detected in any other retinal cell types, subcellular compartments or layers. (c) No NMP is detected in saline-injected control eyes, but native Rds expression is detected beginning at PI-2 (P7), consistent with normal ocular development. Scale bars, 20 μm. N=3-5 mice per group. Abbreviations: OS, outer segment layer; ONL, outer nuclear layer; INL, inner nuclear layer.

FIG. 4. Nanoparticle injection leads to biochemical rescue of the rds^{+/-} phenotype. (a-c) cDNA was collected at PI-30, and message levels of photoreceptor proteins were analyzed by qRT-PCR. (a) CBA-NMP nanoparticle injection leads to a modest increase in Rom-1 message levels, while IRBP-NMP nanoparticle injection increases expression four- to five-fold over levels in uninjected control eyes. (b-c) CBA-NMP and IRBP-NMP nanoparticle injections lead to increases in rod (b) and cone (c) opsins. (a-c); N=3 animals per group. (d-f) Protein levels at PI-30 after nanoparticle injection were examined. Representative SDS-PAGE/Western blots from individual retinas are shown (N=5-6 per group). (d) CBA-NMP and IRBP-NMP nanoparticle injections increase Rds and Rom-1 protein levels (protein load: 20 μg per lane). (e) Substantial increases in rhodopsin protein (Rho) are detected after injection of both CBA-NMP and IRBP-NMP nanoparticles (protein load: 10 μg per lane). (f) No change in S-opsin (S-ops) protein level is detected after nanoparticle injection (protein load: 50 μg per lane). (g) Double immunolabeling for transferred Rds (3B6, green) and cone OSs (S-opsin, red) with nuclear counterstain (DAPI, blue) was performed on frozen sections from PI-30 eyes. Representative cones from two different animals are shown for each treatment. Cones in eyes injected with CBA-NMP express transferred NMP at easily detectable levels (top row). Cones in eyes injected with IRBP-NMP express transferred NMP at variable levels (middle row). Saline injected eyes express no transferred NMP (bottom row). Scale bar, 5 μm; N=3-5 animals per treatment group. Abbreviations: OS, outer segment layer; IS, inner segment layer; ONL, outer nuclear layer.

FIG. 5. Nanoparticle injection leads to partial functional rescue of the rds^{+/-}. A subset of individual injected animals identified from PI-30 ERG analysis (Table 2) was chosen for follow-up. (a-b) Top: representative scotopic traces from naked DNA (gray)- and nanoparticle (black)- injected eyes at PI-30 (a) Scotopic a-wave amplitudes from eyes injected with CBA-NMP nanoparticles are elevated at PI-30, but drop almost back to baseline by PI-60 (b) IRBP-NMP nanoparticle-injected animals retain improved rod function (as measured by scotopic a-wave) through PI-120 (c-d) Top: representative photopic traces from naked DNA (gray)- and nanoparticle (black)-injected eyes at PI-30 (c) Cone function (as measured by photopic b-wave) does not remain substantially improved past PI-30 in eyes injected with CBA-NMP nanoparticles. (d) Photopic b-wave amplitudes in IRBP-NMP nanoparticle-injected eyes remain stable and elevated through the last time point examined (PI-120). Amplitudes from naked DNA-injected eyes are from N=6 animals per group, ± standard error.

FIG. 6. Nanoparticle injection leads to structural rescue of the rds^{+/-} phenotype. (a-b) Light micrographs (top row) and electron micrographs (bottom row, N=3-5 per group) from rds^{+/-} were examined. (a) At PI-30, moderate ultrastructural rescue is detected in the OSs of nanoparticle injected eyes near the site of injection (arrows). (b) By PI-120 significant ultrastructural improvement in OSs of nanoparticle injected eyes is apparent. OS discs are properly aligned and flattened and OS do not exhibit the swirl-like structures typical of the rds^{+/-}. RPE, retinal pigment epithelium; OS, outer segment layer; IS, inner segment layer; ONL, outer nuclear layer. Scale bar, 10 μm.

FIG. 7. Nanoparticle-driven gene expression can precede native gene expression with P2 injection. Rds^{+/-} animals were injected at P2 with nanoparticles or controls (saline or naked DNA) and eyes were harvested and stained at PI-2 for transferred Rds (NMP, 3B6; green) and total Rds (Rds-CT; red) with nuclear counterstain (DAPI; blue). Note that transgene-mediated NMP expression begins by PI-2, while native Rds is not yet expressed.

FIG. 8. Wild-type mice fully recover following subretinal injection at P5 (top). One eye of wild-type mice was injected with saline at P5 and rod (scotopic a,b-wave) and cone (photopic b-wave) functional recovery was measured by ERG at P30. There was no lasting functional deficit as a result of the injection (N=5). On the contrary, in the Rds+/− background, the partially degenerated retinas are more fragile and do not recover completely from the subretinal injection procedure (bottom row).

FIG. 9. The ability of the IRBP nanoparticle to lead to pan-retinal structural rescue was assessed by measuring rows of ONL nuclei and the thickness of OSs. Shown in black are measurements from two individual animals that showed improvement at the site of injection at PI-120. The average of 10 uninjected control eyes is shown by the gray dashed line, ± standard deviation (shaded in gray). N, nasal side; T, temporal side. Six brightfield images of toluidine blue-stained sections were captured from each eye using a Zeiss Axiophot® epifluorescence microscope. Images were 100 μm² in area and were collected both nasally and temporally at distances of 200, 400, and 600 μm from the optic nerve head. Three measurements of OS layer thickness and outer nuclear layer (ONL) rows were taken from each image by an observer masked to sample identity (treatment vs. control group), then averaged. There is an increase in both ONL thickness and OS thickness both on the side of the injection (temporal) and to varying degrees on the opposite side (nasal).

### DETAILED DESCRIPTION OF THE INVENTION

The eye is susceptible to a number of hereditary and/or age related degenerative disorders. The retina contains light sensitive receptors, a complex of neurons, and pigmented epithelium, arranged in discrete layers. In humans, the macula is the portion of the retina that lies directly behind the lens. Cones, the photoreceptor cells responsible for central vision, are heavily concentrated in the macula. Central dystrophies, which affect the macula, include Best's disease, age-related macular degeneration, and Stargardt's macular dystrophy. The peripheral retina is composed mainly of rods, which are responsible for side and night vision. Peripheral degenerative retinal diseases include retinitis pigmentosa, choroidemia and Bietti's crystalline dystrophy.

Macular degenerations are a heterogenous group of diseases, characterized by progressive central vision loss and degeneration of the macula and underlying retinal pigmented epithelium. Age-related macular degeneration (AMD) is the most common form of the disease, affecting an estimated 20% of persons over 75 years of age. AMD is poorly understood in terms of etiology and pathogenesis. The very late onset of the disease has made genetic mapping particularly difficult.

Hereditary peripheral retinopathies are also relatively common. Retinitis pigmentosa (RP), for example, affects approximately 1.5 million people worldwide. Substantial genetic heterogeneity has been observed in this condition, with dozens of chromosomal loci identified (Table 1). For example, mutations in the peripherin/RDS gene (PRPH2) have been linked to retinitis pigmentosa and macular degeneration. A single peripherin/RDS mutation apparently caused retinitis pigmentosa, pattern dystrophy and fundus flavimaculatus, in different family members.

In spite of causal heterogeneity, there is significant clinical similarity among RP subtypes. Common signs and symptoms include early electroretinographic abnormalities, opthalmoscopic findings, and protracted, contiguous expansion of the ring-like scotoma toward the macula, leading to progressively worsening tunnel vision.

As noted above, non-viral delivery methods represent an attractive alternative to viral gene therapy, but historically these approaches have been limited by inefficient entrance of the genetic material into the target cells and by attenuated duration of transgene expression [42, 43]. The present invention is directed to a non-viral gene transfer strategy employing single-molecule nucleic acid nanoparticles, in which plasmid nucleic acid (e.g., DNA) is compacted, for example by polyethylene glycol (PEG)-substituted 30-mer lysine peptides (CK30PEG) as discussed in further detail below. Recently, use of DNA nanoparticles has gained popularity as a gene delivery method because of the versatility, small size, ease of preparation, large vector capacity, stability in nuclease rich environments, and high transfectivity of such nanoparticles [44-48]. Their high transfectivity is due, in part, to the small particle size [49] and also to specific interactions with cell surface nucleolin and subsequent non-degradative trafficking to the cell nucleus [50]. These nanoparticles can successfully transfect both dividing and non-dividing cells, and have been shown to be effective agents, both in experimental models as well as a phase I/IIa clinical trial in cystic fibrosis subjects, in delivering genes of interest to multiple tissues, including the lung, retina, and brain [45, 47, 49, 51-53].

The present invention is a method of using compacted nucleic acid (such as DNA) nanoparticles for non-viral gene transfer to various tissues of the human eye or eyes of other mammals. These nanoparticles comprise, in one embodiment, a neutrally-charged complex containing a single molecule of plasmid DNA compacted with polyethylene glycol (PEG)-substituted polylysine peptides. These complexes are stable in saline and serum, have been shown to efficiently transfect post-mitotic airway cells following in vivo delivery, are non-toxic following lung delivery, and can be repetitively dosed without decrement in biologic activity [24-26]. The size of the expression cassette does not appear to be a limiting factor as plasmids up to 20 kbp have demonstrated cellular transfection and gene transfer [27]. Varying the counterion at the time of compaction can lead to different 3-dimensional shapes of the nanoparticles which can facilitate the development of customized nanoparticles to transfect a multitude of cell types [28]. Clinical studies also have demonstrated the safety and effectiveness of this system in human subjects [29]. Varying the site of injection and type of nanoparticle results in cell-specific transfection. Furthermore, altering the dose of the injected nanoparticles allows fine-tuning to the correct level of gene expression needed for the therapeutic gene.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the subretinal space, the conjunctiva, the subconjunctival space, the episcleral space, the intracorneal space, the epicorneal space, the sclera, the pars plana, surgically-induced avascular regions, the macula, the retina, and the optic nerve.

As used herein, an “ocular condition” is a disease, disorder, or condition which affects or involves the eye or one of the parts or regions of the eye and which is not normal to the subject or animal in a healthy state. Broadly speaking the eye includes the eyeball and the tissues and fluids which constitute the eyeball, the periocular muscles (such as the oblique and rectus muscles) and the portion of the optic nerve which is within or adjacent to the eyeball. The ocular condition or disease may be caused by or due to genetic modifications, such as due to recessive, dominant, autosomal, or X or Y-linked mutations, for example, or trauma, or infections or any other causitive factor, or acquired disorders.

An anterior ocular condition is a disease, disorder, or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, disorder, or condition, such as for example, aphakia; pseudophakia; astigmatism; blepharospasm; cataract; conjunctival diseases and infections; conjunctivitis; corneal diseases; corneal ulcer; dry eye syndromes; eyelid diseases; lacrimal apparatus diseases; lacrimal duct obstruction; myopia; presbyopia; pupil disorders; anterior chamber infections; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, disorder, or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as bacterial, fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, other forms of optic neuropathy and optic neuritis, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

Specific targetable cells within the eye include, but are not limited to, cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, and epithelium of ocular sebaceous glands.

The term “treat”, “treating”, or “treatment” as used herein, refers to reduction or resolution or prevention of an ocular condition, ocular injury or damage, or to promote healing of injured or damaged ocular tissue.

The term “therapeutically effective amount” or “effective amount” as used herein, refers to the level or amount of agent needed to treat an ocular condition, or reduce or prevent ocular injury or damage without causing significant negative or adverse side effects to the eye or a region of the eye.

An “oligonucleotide” or “nucleic acid” according to the present invention may comprise two or more naturally occurring or non-naturally occurring deoxyribonucleotides or ribonucleotides linked by a phosphodiester linkage, or by a linkage that mimics a phosphodiester linkage to a therapeutically useful degree. According to the present invention, an oligonucleotide will normally be considered to be double-stranded unless otherwise obvious from the context, and a nucleic acid may be single stranded or double stranded. The therapeutic oligonucleotide disposed within the nanoparticle may be used to express a desired protein or to function as an anti-sense moiety, and examples include a gene, cDNA, RNA, siRNA, or an shRNA. Additionally, an oligonucleotide or nucleic acid may contain one or more modified nucleotides; such modification may be made in order to improve the nuclease resistance of the oligonucleotide, to improve the hybridization ability (i.e., raise the melting temperature or Tm) of the resulting oligonucleotide, to aid in the targeting or immobilization of the oligonucleotide or nucleic acid, or for some other purpose. The term “nucleic acid” as used herein means either DNA or RNA, or molecules which contain both ribo- and deoxyribonucleotides. The nucleic acids include genomic DNA, cDNA and oligonucleotides including sense and anti-sense nucleic acids. The nucleic acid may be double stranded, single stranded, or contain portions of both double stranded or single stranded sequence.

In addition to the therapeutic nucleic acid, the nanoparticle DNA may also contain DNA sequences either before or after the therapeutic sequence for promoting high level and/or tissue-specific transcription of the nucleic acid in a particular cell in the eye, may promote enhanced translation and/or stabilization of the mRNA of the therapeutic gene, and may enable episomal replication of the transgene in eye cells. The therapeutic gene may be contained within a plasmid or other suitable carrier for encapsulation within the nanoparticle. Alternatively, if the nucleic acid is single or double-stranded RNA, an RNA derivative, or siRNA, such nucleic acids may be directly compacted with polycationic polymers to form nanoparticles. The therapeutic nanoparticle may contain one or more genes, cDNAs, RNAs, shRNA moieties, or siRNAs.

The number of therapeutic genes or nucleic acids encapsulated within the nanoparticle may vary from one, two, three to many, depending on the disease being treated but preferably is one and preferably includes one or more promoters.

In the preferred embodiment, the exogeneous nucleic acid of the nanoparticle used herein encodes a protein to be expressed. That is, it is the protein which is used to treat the ocular disease. In an alternative embodiment, the exogeneous nucleic acid is an anti-sense nucleic acid, which will inhibit or modulate the expression of a protein. In this embodiment, the exogeneous nucleic acid need not be expressed. Thus, for example, ocular tumor cells may express undesirable proteins, and the methods of the present invention allow for the addition of anti-sense nucleic acids to regulate the expression of the undesirable proteins. Similarly, the expression of mutant forms of a protein may cause ocular disease. It is possible to incorporate in the nanoparticle both anti-sense nucleic acid to reduce the level of expression of the mutant endogeneous gene as well as nucleic acid encoding a correct copy of the gene.

In an additional embodiment, the exogeneous nucleic acid of the nanoparticle of the present invention may encode a regulatory protein such as a transcription or translation regulatory protein. In this embodiment, the protein itself may not directly affect the ocular disease, but instead may cause the increase or decrease in the expression of another protein which affects the ocular disease.

In one embodiment, the exogeneous nucleic acid encodes a single protein. In alternative embodiments, the exogeneous nucleic acid encodes more than one protein. Thus, for example, several proteins which are useful to treat an ocular disorder may be desirable; alternatively, several ocular diseases may be treated at once using several exogeneous nucleic acids encoding several proteins.

Similarly, an “exogeneous” or “recombinant protein” is a protein made using recombinant techniques, i.e. through the expression of an exogeneous or recombinant nucleic acid as described above. A recombinant protein is distinguished from naturally occurring protein by at least one or more characteristics. For example, the protein may be made at a significantly higher concentration than is ordinarily seen, through the use of a inducible promoter or high expression promoter, such that increased levels of the protein is made. Thus, for instance, an exogeneous protein is one which may not ordinarily expressed in the ocular tissue. Alternatively, the protein may be in a form not ordinarily found in nature, as in the addition of an epitope tag or amino acid substitutions, insertions and deletions.

In one embodiment for example, the present invention provides non-viral therapies for resolving the genetic abnormalities of ocular diseases associated with mutations in the peripherin/Rds gene (PRPH2) that are known to cause retinitis pigmentosa and macular degeneration in patients. Thus, in a particularly preferred embodiment of the invention directed to treating autosomal dominant forms of retinitis pigmentosa, particles comprising normal forms of the PRPH2 (peripherin/rds) gene are used in the nanoparticles of the invention. The PRPH2 nanoparticles are effective in overcoming ocular deficiencies caused by dozens of mutations which are known to occur in the human PRPH2 gene (as shown for example in Table 1).

The invention provides a method for treating an ocular disorder in a human, other mammalian or other animal subject. In one embodiment, the ocular disorder is one which involves a mutated or absent gene in an ocular cell such as a retinal pigment epithelial cell or a photoreceptor cell. The method of this invention comprises the step of administering to the subject by injection an effective amount of a nanoparticle comprising a nucleic acid sequence encoding an ocular cell-specific normal gene operably linked to, or under the control of, a promoter sequence which directs the expression of the product of the gene in the ocular cells and replaces the lack of expression or incorrect expression of the mutated or absent gene.

Peripherin/rds (P/rds) is an integral membrane glycoprotein distributed along the disc rim region of rod and cone outer segments (OS) as well as adjacent to the connecting cilium at the site of disc morphogenesis. Previous studies have highlighted its necessity in disc assembly, orientation, and physical stability and its suggested role in photoreceptor renewal. Valuable insight into the structural role of P/rds has been provided by the retinal degeneration slow (rds) mouse, in which a lack of endogenous P/rds protein leads to aberrant OS morphogenesis, followed by late-onset retinal degeneration.

As noted elsewhere herein, more than 80 mutations in P/rds have been associated with retinal disease (see Table 1), 70% of which are single-point mutations with the remainder likely leading to a failure in protein expression. The expressed phenotypes caused by these mutations in humans are heterogeneous, including retinitis pigmentosa and cone-rod dystrophy, among others.

TABLE 1
Mutations in the peripherin/RDS protein and gene (PRPH2)

| Phenotype | Mutation | Base change | Nucleotide |
|---|---|---|---|
| Adult vitelliform | Met 1 Thr | ATG > ACG | c.0002 |
| macular dystrophy | | | |
| Digenic RP | Arg 13 Trp | CGG > TGG | c.0037 |
| ADRP | Arg 13 Trp | CGG > AGG | c.0037 |
| Retinitis punctata | 73delTG | AAC TGG > AAC __G | c.0073 |
| albescens, ad | | | |
| COD | Ser 27 Phe | TCC > TTC | c.0080 |
| Age related macular | Ile 32 Val | ATC > GTC | c.0094 |
| dystrophy | | | |
| ADRP | 97insC | ATC ATC > ATC CAT C | c.0097 |
| Adult Onset | 113delG | CTA GGA > CTA _GA | c.0113 |
| Foveomacular | | | |
| Dystrophy | | | |
| Digenic RP | Leu 45 Phe | CTC > TTC | c.0133 |
| ADRP | Leu 45 Phe | CTC > TTC | c.0133 |
| Diffuse retinal | Arg 46 ter | CGA > TGA | c.0136 |
| degeneration | | | |
| ADRP | Arg 46 ter | CGA > TGA | c.0136 |
| ADRP | 198GATGG > AAGACAGA | GGG ATG GGG > GGA | c.0198 |
| | | AGA CAG AGG | |
| Pattern dystrophy | 199delATG | GGG ATG GGG > GGG ___{—} | c.0199 |
| | | GGG> | |
| ADRP | Gly 68 Arg | GGG > AGG | c.0202 |
| Polymorphism | Tyr 83 Tyr | TAC/TAT | c.0249 |
| Pattern dystrophy | 258delACCCAGCC | CTG GAC CCA GCC | c.0258 |
| | | AAG > CTG G________{—} | |
| | | AAG | |
| Polymorphism | Tyr 101 Tyr | TAC/TAT | c.0303 |
| Polymorphism | Val 106 Val | GTC/GTT | c.0318 |
| ADRP | 355delTGC | CTC TGC TGC > CTC | c.0355 |
| | | ___TGC | |
| CRD | 371delG | CTT CGG GGC > CTT C_G | c.0371 |
| | | GGC | |
| CACD | Ser 125 Leu | TCG > CTT | c.0375 |
| ADRP | Leu 126 Arg | CTG > GTG | c.0376 |
| ADRP | Tyr 141 His | TAC > CAC | c.0421 |
| ADRP | Tyr 141 Cys | TAC > TGC | c.0422 |
| Pattern dystrophy | 424insTACT | TAC TAC CGG > TAC TAC | c.0424 |
| | | TAC TCG G | |
| CACD | Arg 142 Trp | CGG > TGG | c.0424 |
| CACD | 441delT | CCT GGC > CC_GGC | c.0441 |
| ADRP | Lys 153 Arg | AAG > AGG | c.0458 |
| Fundus flavimaculatus | 460delAAG | AAG AAG AAC > AAG ___{—} | c.0460 |
| | | AAC | |
| Pattern dystrophy | Asp 157 Asn | GAC > AAC | c.0469 |
| ADRP | Cys 165 Tyr | TGC > TAC | c.0494 |
| Butterfly shaped | Gly 167 Asp | CGG > CAG | c.0500 |
| pigment dystrophy | | | |
| MD | 505delAAC | GGC AAC AAC | c.0505 |
| | | GGT > GGC ___AAC GGT | |
| CRD | Gly 170 Ser | GGT > AGT | c.0508 |
| CACD | Arg 172 Gly | CGG > GGG | c.0514 |
| MD | Arg 172 Trp | CGG > TGG | c.0514 |
| CACD | Arg 172 Trp | CGG > TGG | c.0514 |
| MD | Arg 172 Gln | CGG > CAG | c.0515 |
| ADRP | Asp 173 Val | GAC > GTC | c.0518 |
| RP | Gln 178 Arg | CAG > CGG | c.0533 |
| ADRP | Trp 179 Arg | TGG > CGG | c.0535 |
| Cone-Rod dystrophy | Tyr 184 Ser | TAC > TCC | c.0551 |
| Digenic RP | Leu 185 Pro | CTG > CCG | c.0554 |
| ADRP | Leu 185 Pro | CTG > CCG | c.0554 |
| Pattern Dystrophy | 577delAAA | GTC AAA GA gtgag > GTC | c.0577 |
| | | ___GA gtgag | |
| ADRP | 578delAA | GTC AAA GA gtgag > GTC | c.0578 |
| | | A__GA gtgag | |
| ADRP | Microdeletion | del ex 2/3 | c.0581 |
| CACD | Arg 195 Leu | CGA > CTA | c.0584 |
| Cone-Rod dystrophy | Lys 197 Glu | AAG > GAG | c.0589 |
| ADRP | Val 200 Glu | GTG > GAG | c.0599 |
| MD | 609del17bp | CGG TAC CTG GTG GAC | c.0609 |
| | | GGC GTC > C________{—} | |
| | | __________TC | |
| ADRP | 616delGTGGACGGCGTC | CTG GTG GAC GGC GTC | c.0616 |
| | | CCT > CTG _________{—} | |
| | | ___CCT | |
| CACD | Gly 208 Asp | GCC > GAC | c.0623 |
| Pattern Dystrophy | 624insG | GGC > GGGC | c.0624 |
| ADRP | Pro 210 Ser | CCT > TCT | c.0628 |
| Foveomacular | Pro 210 Arg | CCT > CGT | c.0629 |
| dystrophy | | | |
| ADRP | Pro 210 Leu | CCT > CTT | c.0629 |
| ADRP | Phe 211 Leu | TTC > TTA | c.0633 |
| ADRP | Ser 212 Gly | AGC > GGC | c.0634 |
| Adult vitelliform | Ser 212 Thr | AGC > ACC | c.0635 |
| macular dystrophy | | | |
| Pattern dystrophy | Cys 213 Arg | TGC > CGC | c.0637 |
| Pattern dystrophy | Cys 213 Tyr | TGC > TAC | c.0638 |
| ADRP | Cys 214 Ser | TGC > TCC | c.0641 |
| ADRP | Cys 214 Tyr | TGC > TAC | c.0641 |
| ADRP | Pro 216 Ser | CCT > TCT | c.0646 |
| ADRP | Pro 216 Leu | CCT > CTT | c.0647 |
| ADRP | 656delCAC | TCG CCA CGG > TCG C__{—} | c.0656 |
| | | _GG | |
| MD | Pro 219 Arg | CCA > CGA | c.0656 |
| Pattern dystrophy | Arg 220 Trp | CGG > TGG | c.0658 |
| Pattern dystrophy | Arg 220 Gln | CGG > CAG | c.0659 |
| Adult Vitelliform | 673ins37bp | ins 37 bp | c.0673 |
| Macular Dystrophy | | | |
| ADRP | Gln 226 Asp | CAG > GAG | c.0676 |
| Pattern dystrophy | 701insT | TAC > TTAC | c.0701 |
| Polymorphism | Tyr 236 Tyr | TAC/TAT | c.0708 |
| Pattern dystrophy | Gln 239 ter | CAG > TAG | c.0715 |
| MD | Asn 244 His | AAC > CAC | c.0730 |
| Cone-Rod dystrophy | Asn 244 Lys | AAC > AAG | c.0732 |
| ADRP + Bull's Eye | Asn 244 Lys | AAC > AAA | c.0732 |
| Maculopathy | | | |
| ADRP | Trp 246 Arg | TGG > CGG | c.0736 |
| MD | Tyr 258 ter | TAC > TAA | c.0773 |
| ADRP | Gly 266 Asp | GGT > GAT | c.0797 |
| Adult vitelliform | Val 268 Ile | GTC > ATC | c.0802 |
| macular dystrophy | | | |
| MD | 824delTC | TTC GAG > T__GAG | c.0824 |
| MD | IVS2 + 22ins7bp | ctggg ggtag > ctggg ggtaga | c.0828 |
| | | gggtag | |
| Adult dominant retinal | IVS2 + 3A > T | gtagg > gttgg | c.0828 |
| degeneration | | | |
| Pattern Dystrophy | Tyr 285 ter | TAC > TAA | c.0855 |
| Cone Dystrophy | Ser 289 Leu | TCG > TTG | c.0866 |
| Butterfly shaped | 897delTG | TCT GAG > TC__AG | c.0897 |
| pigment dystrophy | | | |
| Pseudovitelliform | Glu 302 ter | GAG > TAG | c.0904 |
| macular dystrophy | | | |
| Polymorphism | Glu 304 Gln | GAG/CAG | c.0910 |
| ADRP | 914del9bp | AGGGCTGGCTGCTGG > | c.0914 |
| | | AGG_______TGG | |
| Adult vitelliform | Gly 305 Asp | CGG > CAG | c.0914 |
| macular dystrophy | | | |
| ADRP | 920delT | CTG > C_G | c.0920 |
| Polymorphism | Lys 310 Arg | AAG/AGG | c.0929 |
| Polymorphism | Pro 313 Leu | CCG/CTG | c.0938 |
| Adult vitelliform | Trp 316 ter | TGG > TAG | c.0947 |
| macular dystrophy | | | |
| Pattern Dystrophy | Gln 331 ter | CAG > TAG | c.0991 |
| Polymorphism | Gly 338 Asp | GGC/GAC | c.1013 |
| Polymorphism | 1426a/g | aagtt/aaatt | c.1186 |
| Polymorphism | 1587a/g | tacac/tacgc | c.1587 |
| Polymorphism | 1806t/c | catcc/caccc | c.1806 |
| Polymorphism | 1891del4bp | caatc agaca/ca____gaca | c.1891 |
| Polymorphism | 1942a/c | gccaa/gccca | c.1942 |
| Polymorphism | 2045a/c | caaga/ccaga | c.2045 |
| Polymorphism | 2401t/c | ctttg/ctctg | c.2401 |
| Polymorphism | 2419t/c | tagtg/cagtg | c.2419 |

The methods of gene therapy of the present invention are applicable for multiple forms of ocular diseases. As intravitreal injection targets the tissues in the front of the eye, this mode of therapy is widely applicable for corneal diseases such as cataracts and keratoconus. Expression of inflammatory regulators and siRNA via the nanoparticles of the present invention can also be used for treating infectious diseases affecting the cornea [30, 31]. Intravitreal injection can be effective in transfecting retinal ganglion cells whereas optic nerve cells preferably are transfected following subretinal injection. “Acetate” produced (ellipsoidal) nanoparticles for example can be transported in a retrograde fashion to the cell nuclei of optic nerve fibers in the lateral geniculate nucleus. Therefore, as discussed in more detail herein, these methodologies are suitable for treating multiple optic nerve diseases, including optic neuritis, Leber's hereditary optic neuropathy, and glaucoma [32, 33]. Specifically, delivery of brain derived neurotrophic factor (BDNF) had a protective effect in animal models of glaucoma [34].

With the use of nanoparticle-mediated gene transfer contemplated herein, it is possible to have retinal ganglion and optic nerve cells produce substantial levels of BDNF to promote their own sustainability during the stress from intraocular pressure that is observed in glaucoma. For other types of optic nerve injuries, the production of oncomodulin by DNA nanoparticles is a practical approach for regenerating damaged axons [35]. Also, a recent study demonstrated that ectopic expression of a microbial-type rhodopsin in retinal ganglion cells was capable of restoring visual function in mice lacking photoreceptors [36]. Consequently, intravitreal injection to deliver this gene to RGCs via the nanoparticles contemplated herein can be used in treatment of a multitude of retinal degenerative disorders. Furthermore, previous studies using polystyrene nanospheres described the vitreous as a barrier to gene transfer [37], however the specialized compaction procedure utilized for the DNA nanoparticles used herein produced a significantly smaller particle that is freely diffusible through the vitreous.

The results of the present invention using subretinal injection show a dramatic transfection of photoreceptor and RPE cells, demonstrating a significant utility for this non-viral system in rescuing multiple forms of retinal disease. As the present system is capable of delivering large DNA cassettes, it is possible to deliver the entire gene structure in some cases. For many inherited retinal diseases such as retinitis pigmentosa and Stargardt's disease, the disease pathogenesis arising from genetic mutations is understood and various gene therapy strategies have already been developed [4, 11, 15, 38]. Current therapies available for the treatment of the “wet” form of AMD involve the use of small molecules to block the activity of vascular endothelial growth factor, but entail recurring injections to maintain this inhibitory effect [39, 82, 83]. The use of gene transfer using the nanoparticles contemplated herein to deliver an expression cassette to photoreceptors and RPE cells that produces a similar inhibitory molecule is a less invasive strategy as it will produce a more sustained effect. The nanoparticle used herein may comprise any one or more of the genes described herein as long as the nanoparticle functions in accordance with the present invention to transfect the ocular cells as contemplated herein.

### EXPERIMENTAL

The purpose of the present experiments was to test the efficacy of CK30PEG nanoparticles with regard to their ability to rescue the rds^{+/-} adRP-like phenotype thereby showing the effectiveness of this technology for the treatment of human hereditary eye diseases. The rds model is generally recognized as challenging to rescue, because of the severe structural defect associated with the complete absence of Rds protein [63]. We and others have shown that at least 60% of the normal amount of Rds is necessary in order to build photoreceptor Oss [61, 65]. Only one other group has documented partial rescue of an rds model with neonatal gene therapy using an AAV vector [41, 66, 67].

Presented herein are novel results showing that the non-viral DNA nanoparticle vector is capable of achieving significant rescue of the disease phenotype in the rds^{+/-} adRP model.

Results provided in previous work (U.S. Provisional Application 60/927,388) showed that CK30PEG nanoparticles containing a CMV-EGFP plasmid could be used to safely and efficiently transfer genes to the eyes of adult wild-type mice [51]. Close to 100% of retinal photoreceptor cells were transfected and gene expression levels could be titrated to mimic the expression levels of native photoreceptor genes. A murine model of retinitis pigmentosa (the rds^{+/-} mouse) was used for the disease rescue studies of this example. The protein product of this gene, Rds (also called peripherin/rds or peripherin 2), is a tetraspanin glycoprotein known to form homomeric complexes as well as heteromeric complexes with a related tetraspanin protein, rod outer segment membrane protein 1 (Rom-1). Rds is photoreceptor-specific and is critical for photoreceptor disc rim assembly, outer segment (OS) orientation, photoreceptor structural stability, and OS disc renewal [54-56]. Over 80 different mutations in Rds have been identified in humans (see Table 1) and are associated with multiple retinal diseases, including autosomal dominant retinitis pigmentosa (adRP) and progressive macular degeneration (MD) [57-60]. Unlike the retina in the homozygote (rds^{-/-}) mouse, which fails to form OSs and undergoes fairly rapid apoptotic photoreceptor cell death, the retina in the heterozygous (rds^{+/-}) mouse forms OSs, but they are highly disordered, malformed, and short (compared to normal OSs), and exhibits electrophysiological defects and reduced levels of key phototransduction proteins [6-64]. The rds^{+/-} mouse exhibits a classic autosomal dominant RP (adRP) phenotype since haploinsufficency, with reduced levels of Rds protein, results in a disease phenotype. Hence, Rds replacement therapy in the rds^{+/-} mouse represents a suitable, clinically relevant model of retinitis pigmentosa for testing therapeutic intervention.

Nanoparticle Formulation.

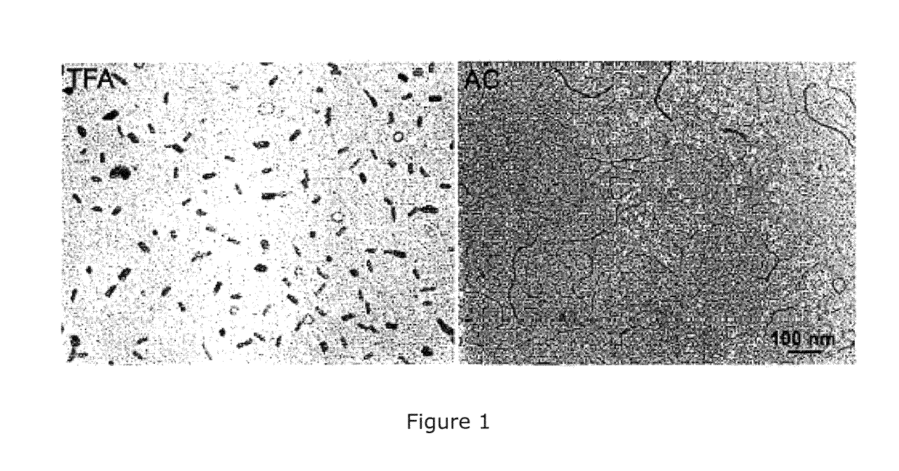

Plasmid DNA was compacted as unimolecular nanoparticles using polylysine peptides having cysteine residue on the N-terminal and thereof. Stability in saline was achieved by covalently modifying the lysine peptide with a PEG molecule or other suitable polymer. A preferred condensing peptide consists of a 30-mer lysine peptide with an N-terminal cysteine, to which (e.g., 10 kDa) PEG is coupled to form a CK₃₀PEG10k molecule. These DNA nanoparticles have a homogenous size and volume distribution (a minor diameter of <20-25 nm for plasmids <20 kb), are stable in saline at concentrations of at least 12 mg/mL of DNA, and are stable in saline for >3 years at 4° C., 9 months at room temperature, and 1 month at 37° C. As visualized by electron microscopy (FIG. 1), these nanoparticles have distinct shape parameters based on the lysine amine counterion present at the time of DNA mixing. For example, the nanoparticles are spheroids or ellipsoids if trifluoroacetate (TFA) is the counterion, whereas rodlike forms are observed if acetate is the counterion. Other counterions, including chloride, bromide, and bicarbonate, may be used to provide the particle with other characteristic shape distributions, including toroids. Other methods which may be used to produce the nanoparticles of the present invention are shown in U.S. Pat. Nos. 5,844,107; 5,877,302; and 6,281,005, for example. Peptides used may be from 8-30 mer and preferably comprise lysine and/or arginine. Nanoparticles were concentrated up to 4 mg/ml of DNA in saline. Minor diameters of both types of nanoparticles are <25 nm since the size of the nuclear membrane pore through which the nanoparticle must pass has a diameter of 25-27 nm. Examples of polymers other than PEG 10 kd which can be used to form the nanoparticles used in the present invention include, but are not limited to, those described in U.S. Pat. Nos. 5,844,107; 5,877,302; 6,008,336; and 6,077,835, and methods and apparatus for making the compacted nanoparticles used in the present invention are described in U.S. Pat. Nos. 6,281,005 and 6,506,890. Other publications which describe the construction and composition of compacted nanoparticles contemplated for use in the present invention include, but are not limited to, U.S. Published Applications 20020042388, 20030078229, 20030078230, 20030134818, 20030171322, and 20040048787.

Mice.

All experiments and animal maintenance were approved by the local Institutional Animal Care and Use Committee (Oklahoma City, Okla., U.S.A.) and conformed to the guidelines on the care and use of animals adopted by the Society for Neuroscience and the Association for Research in Vision and Opthalmology (Rockville, Md., U.S.A.). Balb/cJ mice were obtained from Jackson Labs (www.jax.org) and used for all experiments.

Material and Methods.

Construct generation and nanoparticle preparation. Two constructs were generated expressing full-length, wild-type Rds cDNA (1.7 kb) containing the P341Q silent mutation (called NMP-SEQ ID NO:1), which enables specific detection with the 3B6 monoclonal antibody [55]. Either the human inter photoreceptor retinoid-binding protein (IRBP) promoter (1.3 kb) [69] or chicken beta-actin (CBA) promoter (280 bp) (both known and available in the art) was used to drive gene expression. The two promoter regions were amplified from genomic DNA by PCR and sub-cloned into the pXL-TOPO vector in front of NMP using EcoR I and BamH I restriction enzymes. The two plasmid DNAs were individually compacted into rod-like nanoparticles (preferably using acetate as a counterion) at Copernicus Therapeutics as reported previously [46, 52] and as described elsewhere herein and were used at a final concentration of 3.06 μg/μl in 0.9% saline.

Subretinal Injections.

Rds^{+/-} pups at P5 were anesthetized by incubation on ice for 2-2.5 minutes. The eyelid of the right eye was cut, the cornea was exposed, and a puncture in the cornea was made with a 30-gauge needle. A 35-gauge blunt-end needle attached to a 10 μl Nanofil® syringe (World Precision Instruments, Sarasota Fla.) as inserted into the puncture under an operating microscope (Carl Zeiss Surgical, Inc., NY). A volume (0.3 μl) of solution containing fluorescein dye and either nanoparticles, saline (vehicle), or naked plasmid DNA (3.06 μg/μl) was delivered into the subretinal space, usually in the superior temporal quadrant. After injection, the needle was left in place for 3-5 seconds to allow full treatment delivery before being withdrawn gently. Successful delivery of material was confirmed by observation of the fluorescein at the time of injection. The cut eyelid was returned to its original position and the surface of the eye was gently blotted with a Kimwipe. Animals were warmed on a 37° C. bed until fully awake. We have previously shown that this injection technique does not alter ocular development in wild-type mice. All nanoparticles and naked DNA were used at the same concentration (3.06 μg/μl), selected based on data from our previous study [51]. If material delivery could not be confirmed, or if microophthalmia or intraocular infection was observed, the injection was considered unsuccessful and the animal was removed from the study ( 121/432˜28%). Mice were maintained in the breeding colony under cyclic light (14-hour light/10-hour dark) conditions; cage illumination was approximately 7 foot-candles during the light cycle. All procedures were approved by the University of Oklahoma Health Science Center Institutional Animal Care and Use Committee (IACUC) and adhered to the ARVO Statement for the Use of Animals in Ophthalmic and Visual Research (www.arvo.org).

RNA Isolation and qRT-PCR.

Both injected and uninjected eyes were collected at PI-2, 7, 14, 21, and 30 days for analysis of mRNA levels. qRT-PCR was performed with a MyIQ single-color qRT-PCR machine (Bio-Rad), using at least three injected and three uninjected eyes from each treatment group at each of the scheduled time points. Mice were euthanized, eyes were enucleated and homogenized and total RNA was extracted using TRIzol (Invitrogen Inc. Carlsbad, Calif.) as described previously [51]. Subsequently, DNase treatment was performed with RNase-free DNase I (Promega Inc.) to remove both genomic DNA and any remaining nanoparticle DNA. cDNA synthesis by reverse transcription was performed and 20 ng of cDNA from each sample was used for qPCR. qPCR primer sequences were reported previously [51]. Melting curve analysis and agarose gel electrophoresis were performed at the end of the reaction to ensure that the PCR products were specific and of appropriate size. All experimental mRNA levels were quantified against the housekeeping gene hypoxanthine phosphoribosyltransferase (HPRT) as described previously (ΔcT) [51, 78]. Relative expression levels were calculated by 2^{-ΔcT} method [78]. Each sample was run in triplicate in two independent qPCR reactions. To confirm that Rds levels were not artificially altered by the presence of undigested nanoparticle, control reactions amplifying from the IRBP or CBA promoter regions were performed and no product was detected.

Immunohistochemistry.

Whole eyes were enucleated and fixed with phosphate-buffered saline containing 4% paraformaldehyde at 4° C. over night. With the exception of PI-2 eyes, the cornea and lens were removed and the eye was returned to fixative for an additional two hours. The eyes were cryoprotected by serial immersion in 15% and 30% (w/v) sucrose solutions for at least two hours each. Individual eyes were embedded in M1 embedding medium (Thermo Electron Corporation, PA) and frozen on dry ice; frozen sections (10 μm thickness) were cut with a cryostat (Leica) and collected on precleaned Superfrost-plus® microscope slides (Fisher Scientific). For immunohistochemistry, all steps were carried out at room temperature as described previously [51, 77]. The following primary antibodies were used (at 1:100 dilution): 3B6 mAb recognizing NMP (a kind gift from Dr. R. S. Molday, University of British Columbia, Vancouver, BC, Canada), Rds-CT recognizing both NMP and endogenous Rds (generated in-house), and anti-S-opsin recognizing short wavelength mouse opsin (also generated in-house). Antigens were recognized by incubation with FITC-conjugated goat anti-mouse or CY3-conjugated goat anti-rabbit IgGs (Jackson ImmunoResearch Laboratories, Inc.). Staining controls included eyes from age-matched stable NMP transgenic mice and wild-type mice, and slides on which primary or secondary antibodies were omitted. Observation and imaging were performed using an epifluorescent microscope (AxiophotZeiss Ltd., Germany) and a spinning disk confocal microscope (BX62 Olympus, Japan).

Protein Detection By Western Blot.

Western analysis was performed as reported previously [61, 79, 80]. Dissected individual retinas were homogenized on ice and solubilized (50 mM Tris, pH 7.8, 100 mM NaCl, 5 mM EDTA, 0.05% SDS, 1 mM PMSF, 1% TX-100, 2.5% (v/v) glycerol) for one hour at 4° C., then processed for SDS-PAGE and subsequent Western blotting using 10-50 μg protein per lane (as detected by Bradford assay; Bio-Rad). Primary antibodies were used as follows: mAb 1D4, recognizing rhodopsin (at 1:5000; another generous gift from Dr. R. S. Molday); Rds-CT (at 1:1000); anti-S-opsin (at 1:000); Rom-1 (at 1:1000; generated in-house); and β-actin-HRP (at 1:5000; Sigma/Aldrich). Detection of primary antibody binding was performed using HRP-conjugated goat anti-mouse or goat anti-rabbit antibodies and imaged using a Kodak Image Station 4000R with Kodak MI software.

Electroretinography.

Full-field ERG was performed as previously reported [74]. Briefly, mice were dark-adapted overnight, then anesthetized, vibrissae were trimmed and eyes were dilated. Needle electrodes placed in the cheek and the tail of the animal served as reference and ground leads, respectively, while platinum loop electrodes were placed on the cornea for measurements. Scotopic ERGs were recorded using a strobe flash with an intensity of 137-cd s/m², while photopic ERGs were measured by averaging recordings from 25 77-cd s/m² intensity strobe flashes after five minutes of light exposure at 29.0285 cd s/m² (GS-2000, Nicolet Instrument Corp., Madison, Wis.). Analysis was performed as described [61, 74].

Histology and Electron Microscopy.

Enucleated eyes were fixed and sectioned as described previously [81]. Briefly, the superior cornea was marked, and then the eye was enucleated and fixed in 0.1 M sodium cacodylate buffer containing (w/v) 2% glutaraldehyde, 2% paraformaldehyde and 0.025% CaCl₂ at 4° C. overnight. Following removal of the cornea and lens the eyes were post-fixed in 1% (w/v) 0504 in 0.1 M sodium cacodylate at room temperature for one hour. Eyecups were rinsed twice in the same buffer, then embedded in Spurr's resin. Semithin (0.75 μm thickness) sections were stained with 1% (w/v) toluidine blue in 1% (w/v) sodium borate, coverslipped, and viewed with an Olympus BH-2 microscope. Digital images were captured using a Nikon DXM1200 digital camera and stored on a computer, using imaging software. Ultrathin (silver-gold) sections were stained with 2% uranyl acetate and lead citrate and imaged using a JEOL 100CX electron microscope (80 KeV).

Statistical Analyses.

For qRT-PCR data, values are expressed as mean relative expression (±S. D.). For ERG data, nanoparticle-injected groups were compared with naked DNA-injected groups. All groups passed the Kolmogorov-Smirnov test for normality and P-values are from two-tailed, unpaired Student's t-tests. In cases where unequal variance was found (via F-test), Welch's correction was applied. Means and standard errors are reported in Table 2 as indicated.

Rds Nanoparticles Drive High and Persistent Transgene Expression.

In the normal rodent retina, Rds expression and localization to the distal connecting cilium in the rod photoreceptor cell begin around postnatal day 5 (P5) [63, 68] (i.e., before OS formation), and P5 represents a time that precedes the onset of retinal degeneration in the rds mouse model. Hence, we selected P5 as the physiologically appropriate developmental time for therapeutic intervention. Two vectors were generated, each expressing the full length cDNA of normal mouse peripherin/Rds (NMP), one under the control of the ubiquitously expressed chicken beta-actin promoter (CBA), and the other employing the photoreceptor-specific, human interphotoreceptor retinoid-binding protein promoter (IRBP) [69]. Nanoparticles or controls (naked plasmid DNA or saline) were injected subretinally into rds^{+/-} mice at P5 and followed for up to four months.

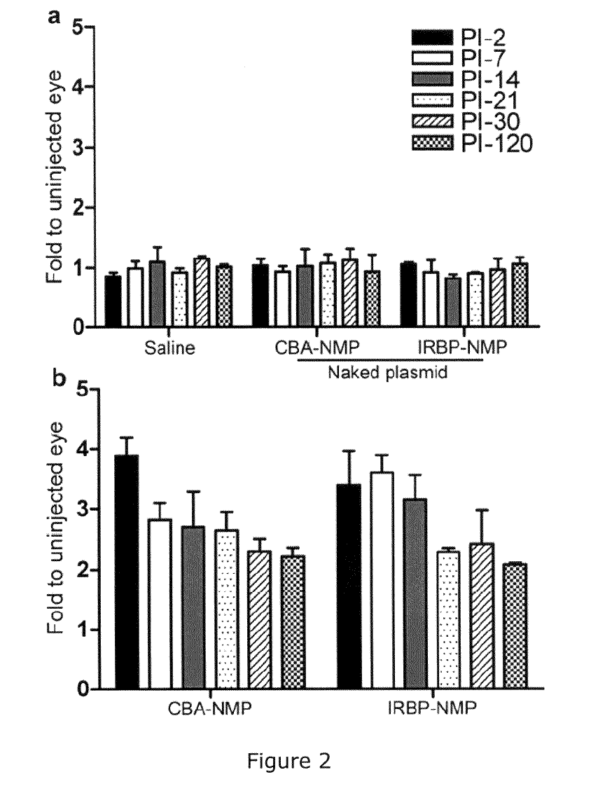

As shown in FIG. 2, injection of both CBA-NMP and IRBP-NMP nanoparticles resulted in expression of Rds message at levels several times greater than in uninjected controls, as measured by qRT-PCR. At post-injection day 2 (PI-2), mRNA levels in CBA-NMP and IRBP-NMP nanoparticle injected eyes (FIG. 2b) were at least three- to four-fold higher than the saline or naked DNA-injected control eyes (FIG. 2a). Eyes injected with IRBP-NMP maintained elevated expression until PI-14, then stabilized levels two- to three-fold higher than controls, while CBA-NMP stabilized at similar levels at PI-7. Neither saline nor naked plasmid DNA produced a significant alteration in Rds mRNA levels, compared to uninjected control eyes (FIG. 2a). Elevated mRNA levels were maintained for up to four months (PI-120), the longest time point examined.

Expression of Exogenously Delivered Gene Occurs in Virtually all Photoreceptors.

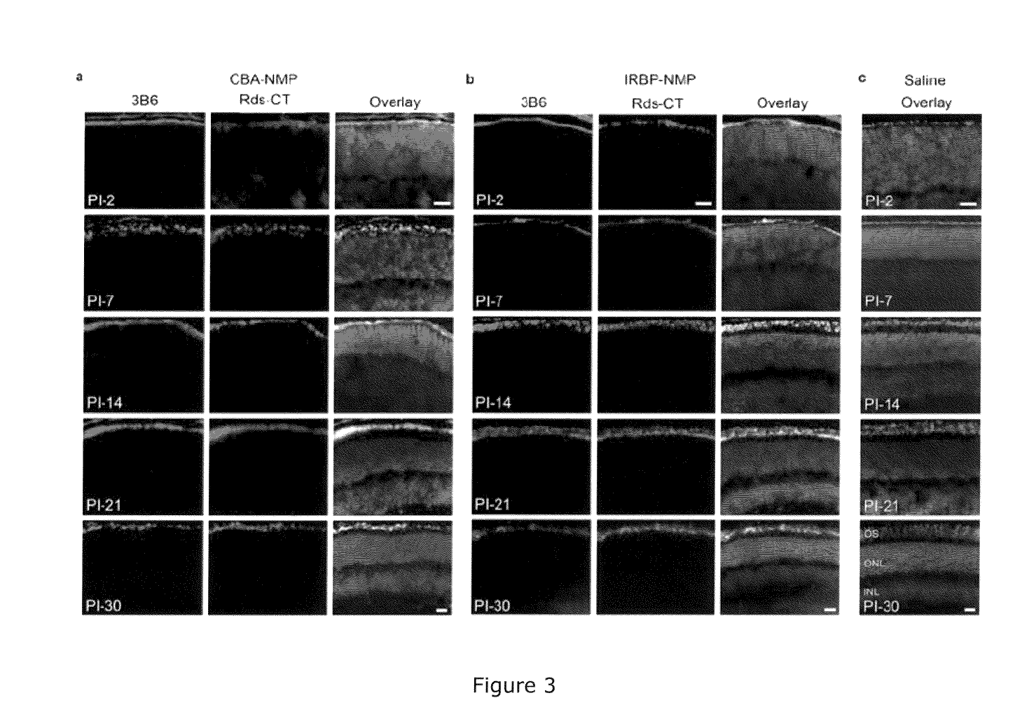

We next examined the identity of the cells that took up the exogenously delivered NMP cDNA and the efficiency of gene product expression within the retina over time, using immunohistochemistry. Due to an epitopic modification in the NMP carboxy terminus (P341Q), the transferred Rds gene product can be detected selectively even on a normal Rds background using the 3B6 monoclonal antibody [61]. The P341Q alteration does not result in retinal disease or vision loss [60]. In contrast, endogenous Rds protein is labeled with the Rds-CT antibody. We have shown that the Rds-CT antibody is also capable of recognizing transgenic NMP, but with much lower efficiency than endogenous (due to the C-terminal modification in NMP). Although normal OS development has not yet begun at PI-2 [70], FIG. 3 (top row) shows expression of both transferred (FIGS. 3a, b) and native Rds (FIG. 3c) protein in the tip of the connecting cilium. By PI-7 (P12), distinct outer and inner nuclear layers are apparent and NMP/Rds staining in nascent OSs is visible as a thin layer adjacent to the photoreceptor nuclei. NMP distribution in the OSs persisted through the latest time point examined (PI-30). NMP also co-localized with native Rds and was limited to the OS layer (FIG. 3); no NMP was detected in any other retinal cell types or ocular tissues, nor in eyes injected with naked DNA (not shown). As expected, NMP expression levels in the OSs were heterogeneous, with stronger signal in some areas than others. However, we estimate that over 95% of photoreceptors expressed the product of the transferred gene. The choice of promoter did not have any apparent effect on cellular distribution: both CBA-NMP and IRBP-NMP nanoparticles exhibited similar distribution patterns at all time points.

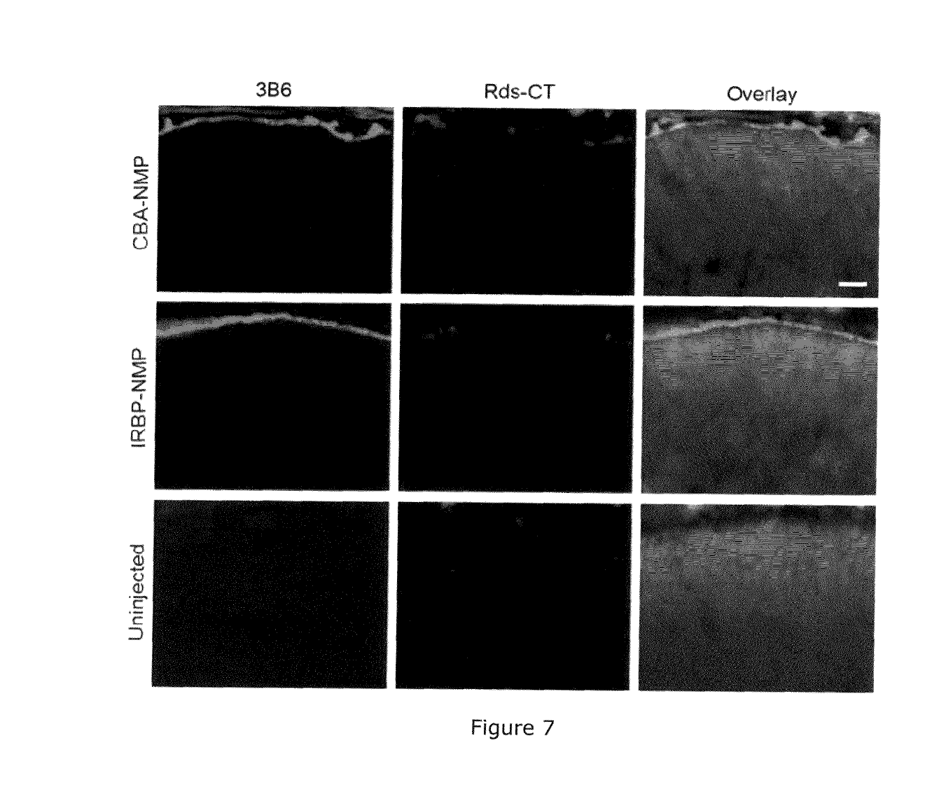

In a particularly surprising result, these nanoparticles are capable of driving transgene expression before native expression begins. When subretinal injections were performed at P2, instead of P5, NMP expression was detected at PI-2 (P4), whereas native (endogenous) Rds expression did not occur that early in development (see FIG. 7).

Rds nanoparticles improve expression levels of key visual transduction proteins.

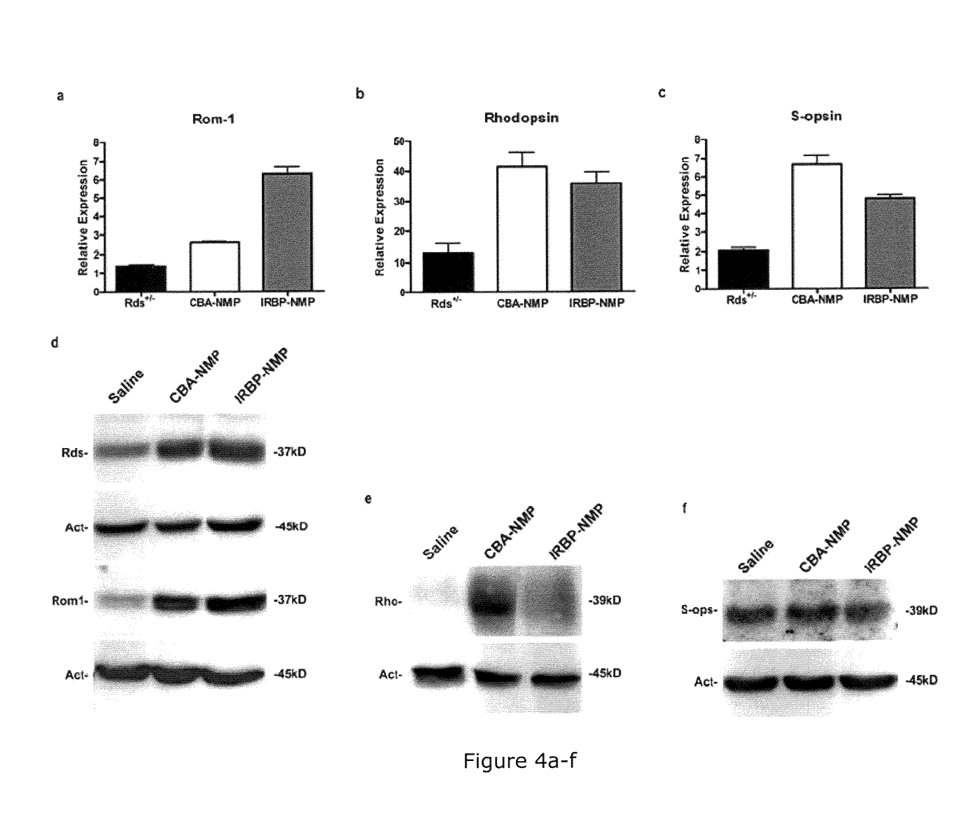

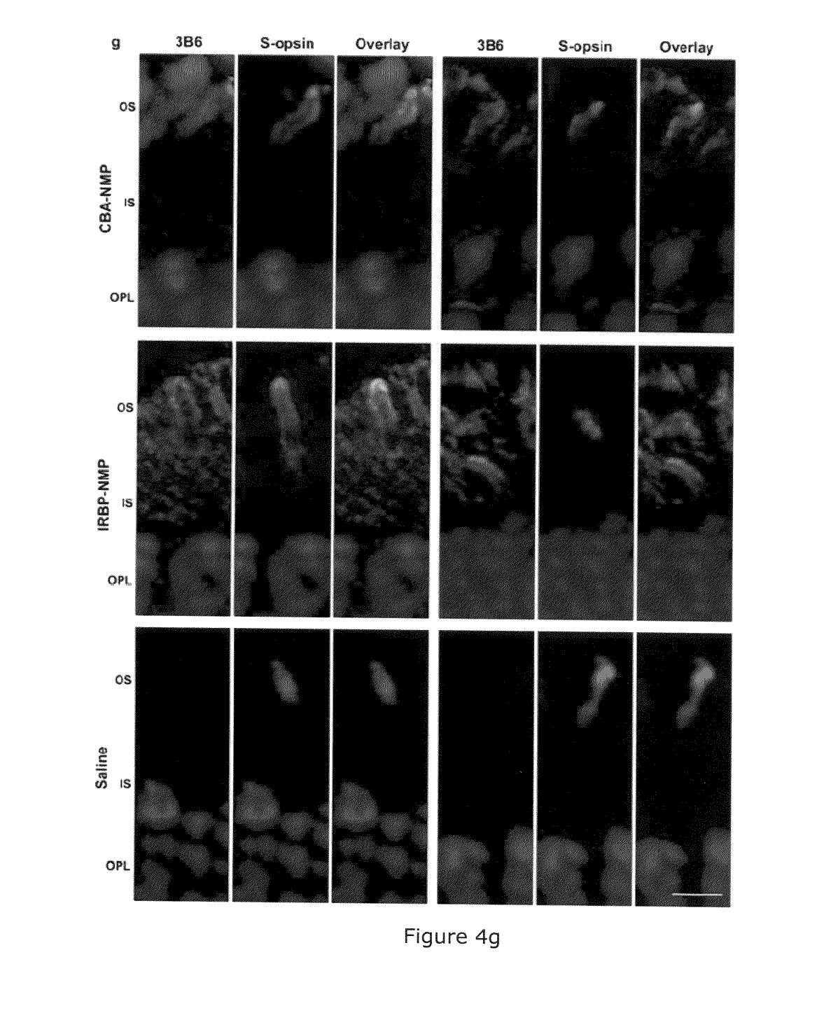

Next, it was determined whether or not nanoparticle-driven expression of NMP results in rescue of the rds^{+/-} disease phenotype. To measure biochemical rescue, we assayed the levels of several photoreceptor-specific proteins known to be decreased by Rds deficiency. FIGS. 4a and 4d show that, at PI-30, expression levels of the Rds binding partner Rom-1 were increased, both in terms of message (by qRT-PCR) and protein (Western blot), compared to uninjected controls. Consistent with the mRNA data presented in FIG. 2, expression of Rds protein was also increased in NMP nanoparticle-injected eyes. Expression of rhodopsin (the rod visual pigment) is necessary for phototransduction and proper photoreceptor maintenance, and is significantly decreased in the rds^{+/-} retina [71]. In FIG. 4, we show that injection of NMP nanoparticles led to increased rhodopsin message (FIG. 4b) and protein (FIG. 4e) expression levels. We also observed a similar increase in the message level of short-wavelength cone opsin (S-opsin, FIG. 4c) after nanoparticle injection, although no alteration in S-opsin protein level was detected (FIG. 4f).

Since the photoreceptor population in the mouse retina consists of 95-97% rods [72, 73], the results presented in FIG. 3 are consistent with the conclusion that the two types of nanoparticles drove gene expression in rods and that their products were delivered with fidelity to the OS. However, it was not clear from those data whether exogenous Rds was expressed in cones. Therefore, double labeling for NMP and S-opsin was performed on PI-30 eyes. Two representative cones from each nanoparticle-injected and control eye are shown in FIG. 4g (single 0.5 μm slices of spinning disk confocal image stacks). Although expression was heterogeneous, most cones from nanoparticle-injected eyes expressed exogenous NMP. As expected, no NMP was expressed in saline-injected eyes (FIG. 4g, bottom row).

Nanoparticle-Driven Rds Expression Restores Visual Function.

The rds^{+/-} mouse RP model exhibits reduced ERG responses indicative of early-onset slow rod degeneration followed by late-onset slow cone degeneration [63, 74]. In order to assess functional rescue of this phenotype after treatment, full-field ERGs were performed on nanoparticle-injected and control mice. Initial ERGs were performed at PI-30 (see Table 2). Average scotopic a-wave amplitudes, indicative of rod function, were increased with statistical significance after injection of either CBA-NMP or IRBP-NMP nanoparticles, compared to amplitudes from eyes injected with naked plasmid DNA. Interestingly, cone function was similarly improved, although only CBA-NMP nanoparticle-injected eyes demonstrated a statistically significant increase overall. The magnitude of rescue varied considerably with both nanoparticles, most likely due to variations in the post-injection recovery, particle delivery, or particle uptake. Several individual nanoparticle-injected animals exhibited significantly greater-than-average rescue. IRBP-NMP and CBA-NMP nanoparticles both mediated up to 150% increase in scotopic a-wave amplitudes, compared to uninjected controls. Maximum cone rescue was also substantially higher than average; several nanoparticle-injected animals had maximum cone amplitudes 130-190% higher than observed in naked DNA-injected controls.

TABLE 2
Table 2 Average full-field ERG values at various timepoints.

| Nanoparticle | | Naked DNA | | | |
|---|---|---|---|---|---|
| Average^{a} ± SEM | #^{c} | Average^{a} ± SEM | #^{c} | % Change^{b} | P^{b} |

PI-30

| | | | | | | | |
|---|---|---|---|---|---|---|---|
| Scotopic-A | CBA-NMP | 134.8 ± 13.3 | 19 | 92.9 ± 9.4 | 6 | 45.1% | 0.018 |
| | IRBP-NMP | 143.6 ± 13.2 | 16 | 93.9 ± 9.2 | 9 | 52.9% | 0.0055 |
| Photopic-B | CBA-NMP | 148.1 ± 11.3 | 19 | 98.2 ± 15.2 | 6 | 50.8% | 0.035 |
| | IRBP-NMP | 131.6 ± 13.8 | 16 | 107.5 ± 15.4 | 9 | 22.4% | 0.27 |
| Scotopic-A | WT | 448.6 ± 27.6 | 10 | | | | |
| Photopic-B | | 167.0 ± 17.2 | 10 | | | | |

PI-60

| | | | | | | | |
|---|---|---|---|---|---|---|---|
| Scotopic-A | CBA-NMP | 107.7 ± 7.8 | 5 | 81.8 ± 17.2 | 4 | 31.7% | 0.0614 |
| | IRBP-NMP | 148.0 ± 11.2 | 8 | 70.5 ± 18.8 | 4 | 110.0% | 0.0037 |
| Photopic-B | CBA-NMP | 117.3 ± 19.7 | 5 | 131.0 ± 11.3 | 4 | −11.5% | 0.5935 |
| | IRBP-NMP | 204.0 ± 16.8 | 8 | 64.5 ± 20.4 | 4 | 216.0% | 0.0005 |
| Scotopic-A | WT | 407.1 ± 50.9 | 14 | | | | |
| Photopic-B | | 184.4 ± 26.9 | 14 | | | | |

PI-120

| | | | | | | | |
|---|---|---|---|---|---|---|---|
| Scotopic-A | CBA-NMP | 123.9 ± 12.9 | 5 | 77.1 ± 17.28 | 6 | 60.7% | 0.086 |
| | IRBP-NMP | 129.4 ± 9.6 | 5 | 79.9 ± 13.4 | 5 | 61.9% | 0.018 |
| Photopic-B | CBA-NMP | 120.8 ± 14.83 | 5 | 108.0 ± 13.48 | 6 | 11.0% | 0.54 |
| | IRBP-NMP | 185.6 ± 20.6 | 5 | 79.6 ± 24.2 | 5 | 133.2% | 0.012 |
| Scotopic-A | WT | 370.6 ± 14.3 | 8 | | | | |
| Photopic-B | | 204.9 ± 24.5 | 8 | | | | |

^{a}Values are mean μV ± S.E.M.
^{b}Comparison between nanoparticle and naked DNA using 2-tailed un-paired Student's T-test as described in methods.
^{c}Number of animals tested.

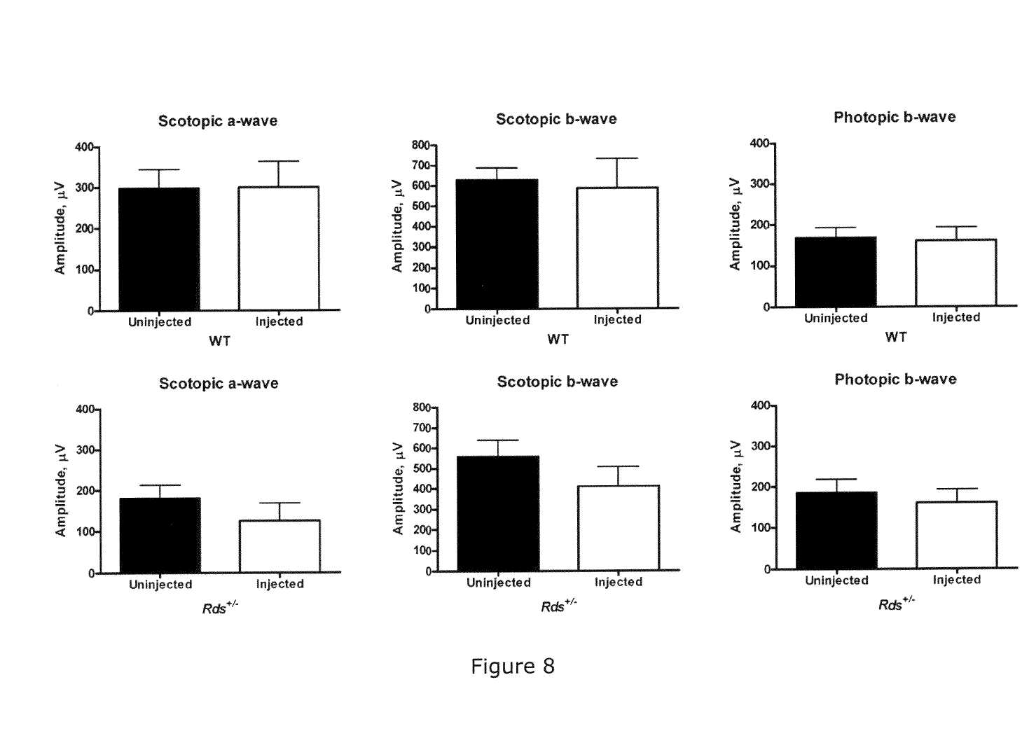

Although we found that the wild-type eye can completely recover from P5 subretinal injection (see FIG. 8—top), we did observe that ERG amplitudes from saline- and naked DNA-injected eyes tended to be lower than in uninjected eyes (FIG. 8—bottom). These data, in combination with our earlier work on adult rds^{+/-}[75], suggest that the rds^{+/-} eye is more fragile than the normal eye and that subretinal injections per se in the mutant may cause adverse effects on visual function which must be overcome by any treatment.

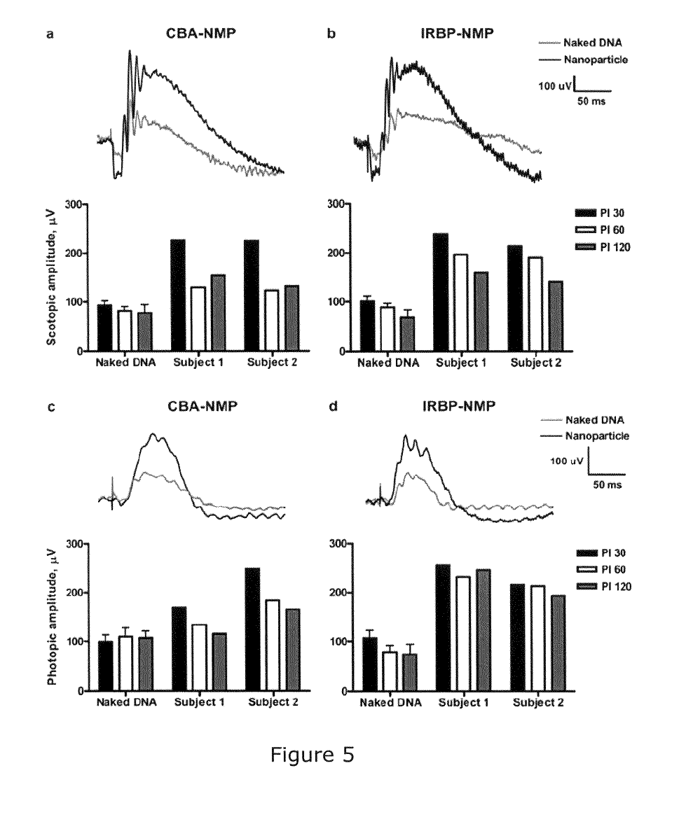

In order to determine whether functional rescue persisted at later timepoints, animals that demonstrated rescue at PI-30 were selected for follow-up at PI-60 and PI-120 (Table 2). Results from two representative animals from each treatment group are shown in FIG. 5. Injection of CBA-NMP nanoparticles did not result in long-term functional rescue of rods (Table 2, FIG. 5a, bottom) or cones (Table 2, FIG. 5c, bottom). Although ERG amplitudes were improved at PI-30, levels declined almost back to baseline when assessed at PI-60 and PI-120. In striking contrast, IRBP-NMP led to substantial improvements in cone and rod function at both PI-60 and PI-120 when compared to naked DNA-injected controls (FIGS. 5b and 5d, bottom). Scotopic a-wave amplitude values for IRBP-NMP-injected animals did decrease gradually over time, but stayed considerably higher than baseline. In some IRBP-NMP-injected eyes, the scotopic a-wave amplitudes at PI-60 were higher than those of uninjected eyes at PI-30 (average uninjected PI-30:168.2±9.85 μV vs. IRBP-NMP PI-60: subject 1, 196.5 μV, subject 2 190.4 μV).

Long-term improvement of cone function in IRBP-NMP nanoparticle-injected eyes was even more significant: photopic b-wave amplitudes did not significantly decrease over time (between PI-60 and PI-120, p=0.51) and in some cases exceeded those of wild-type animals (treated subject 1, 245.6 μV vs. age-matched wild-type average from Table 2 204.5 μV). This suggests that IRBP-NMP nanoparticle-mediated NMP expression is capable of overcoming damage due to subretinal injection per se and can slow or rescue the functional degeneration associated with Rds haploinsufficiency.

OS Ultrastructure is Substantially Improved by Increased Rds Expression.

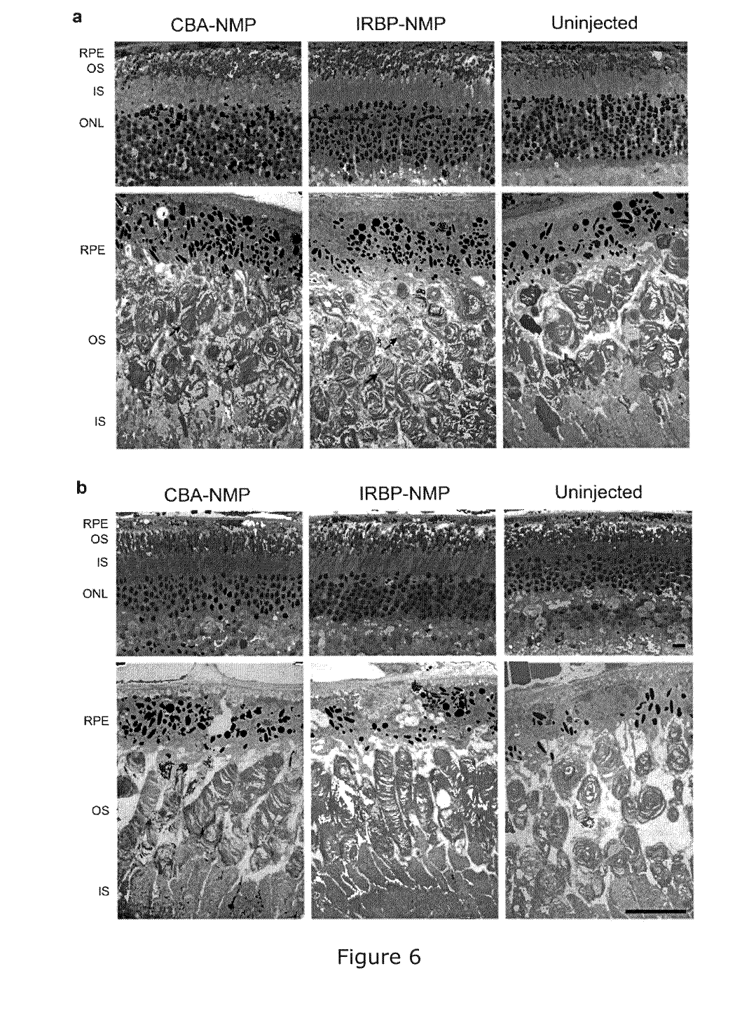

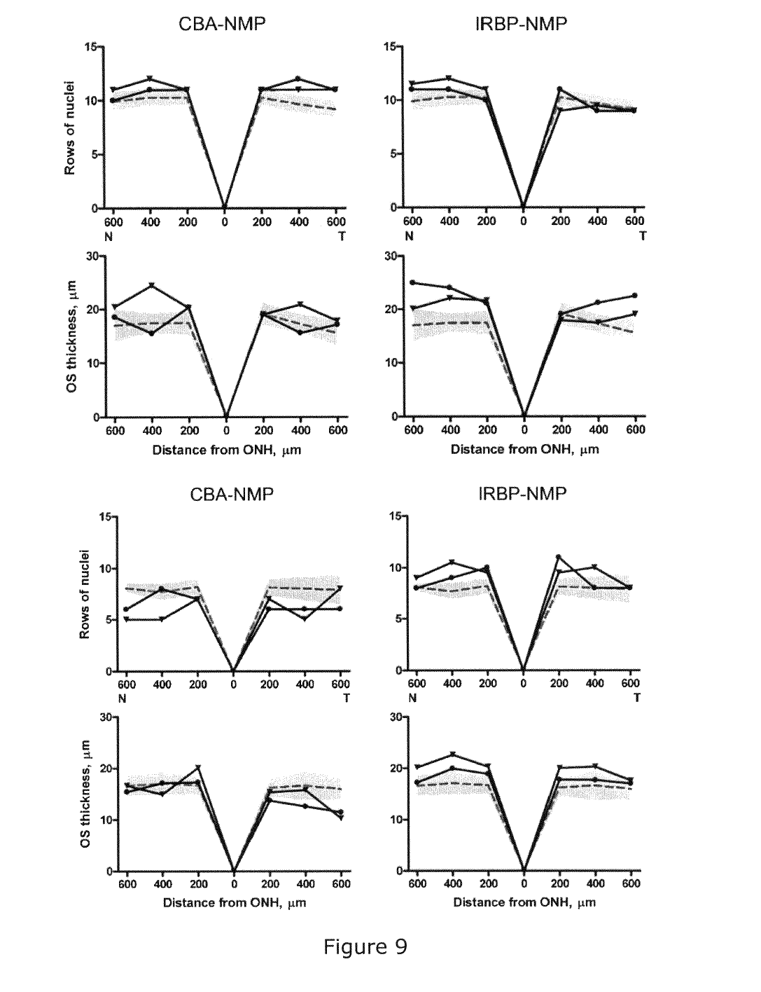

Finally, we analyzed NMP nanoparticle-mediated structural rescue of photoreceptors in the rds^{+/-} retina, using both light and electron microscopy, at PI-30 and PI-120, in comparison with uninjected controls. Photoreceptors in the rds^{+/-} retina typically exhibit very short OSs with misaligned and whorl-like disc membranes. At PI-30, there was a modest increase in outer nuclear layer thickness (FIG. 6a, top), and many individual OSs exhibit improved ultrastructure (arrows, FIG. 6a, bottom). By PI-120, however, virtually all photoreceptors examined near the site of injection showed noticeable structural improvement (FIG. 6b). Consistent with the ERG results (see FIG. 5), structural rescue was more pronounced in the IRBP-NMP-injected eyes compared to CBA-NMP-injected eyes at PI-120, but both exhibited OSs with neat stacks of disc membranes. In addition, pan-retinal analysis of OS thickness and the number of rows of nuclei in the ONL suggests that IRBP-NMP may be capable of driving structural rescue across the retina, not just near the site of injection (FIG. 9).

The present invention is the first demonstration of treatment of an ocular disease phenotype using DNA nanoparticles for gene delivery. These results show that subretinal injection of compacted DNA nanoparticles carrying Rds cDNA at P5 results in gene expression that is: a) high (levels up to four-fold higher than native), b) widely distributed (detected in virtually all photoreceptors), and c) persistent (expression detected up to PI-120, the latest time point examined). Nanoparticle injection also improved expression of key photoreceptor proteins known to be reduced in the rds^{+/-} mouse RP model. Notably, IRBP-NMP nanoparticles afforded significant, persistent (up to PI-120) restoration of both rod- and cone-mediated vision, with full-field cone ERG amplitudes approaching those seen in wild-type mice. Ultrastructural rescue in nanoparticle-injected eyes was similarly pronounced; at four months post-injection, IRBP-NMP animals exhibited properly oriented OSs with nicely stacked discs.

Viral gene therapy has been remarkably successful in treating some types of ocular diseases, e.g., MV-mediated long-term rescue of vision in Briard dogs harboring a mutation in RPE65 [40]. However, since viral vectors have a number of significant limitations, the development of effective non-viral vectors is essential for improved efficacy and safety of gene therapy approaches. A number of non-viral approaches have been explored, including the use of liposomes, electroporation of naked DNA, and gene delivery with dendrimers, yet they have encountered persistent problems with limited uptake and short-term gene expression [43]. The present invention demonstrates the efficacy of compacted DNA nanoparticles comprised of PEG-substituted lysine peptides for gene delivery, as applied to the rds^{+/-} mouse RP model. Because of the structural defects that accompany Rds mutations or deficiency in vivo, rescue of the disease phenotype heretofore has been particularly difficult [41, 66, 67]. However, since most Rds-associated RP in humans is due to loss-of-function mutations causing a haploinsufficiency phenotype, the Rds^{+/-} is directly relevant, and therefore extremely important model to target.

Results herein involve both a ubiquitously expressed promoter and a tissue-specific promoter. Based on previous studies using these promoters in the eye, it was hypothesized that the IRBP promoter would drive expression in rods and cones [69], while the CBA promoter would direct expression in multiple ocular cell types. CBA can drive GFP expression in most ocular tissues after P0 injection into the rat eye, but it has been shown that when a tissue-specific transferred gene is expressed under the control of the CBA promoter, tissue distribution is limited [76]. Our study confirmed this latter point; the product of CBA-NMP driven transgene expression was only detected in photoreceptor OSs, not in other retinal or ocular cell types. The OS-specific distribution of CBA-driven NMP expression is likely due largely to the site of injection (subretinal, as opposed to intravitreal) as well as possibly the rapid turnover of any ectopically expressed protein.

IRBP-mediated NMP expression was only detected in rods and cones, but immunohistochemistry revealed that while most cones express the transferred gene, some do not. The reason for this variation is not known, but it is possible that cones differentially express the nucleolin cell-surface protein known to mediate uptake of the nanoparticles [50]. In spite of variation in nanoparticle-driven gene expression in cones, we see complete functional cone recovery (to WT levels) in many IRBP-NMP treated animals. This is likely because the functional deficit in cones of the Rds^{+/-} is less severe than the rod functional deficit in this model. Indeed we have shown that cones have a different requirement for Rds than rods [77], and that less Rds is needed for cones to form fully functional OS than rods [61]. Due to the more pronounced functional and structural rescue in eyes treated with IRBP-NMP nanoparticles (compared to CBA-NMP), we conclude that IRBP is a superior promoter to CBA for this application.

We chose to treat our animals at P5 both because P5 represents a physiologically appropriate intervention time and because previous gene therapy rds trials have reported difficulties correcting the ERG defect in adult rds mice whereas correction was observed following neonatal gene transfer [66, 76]. The high rates of transfectivity described herein after P5 injection of rds^{+/-} mice combined with the high rates of transfectivity we previously reported after subretinal injection of adult wild-type mice [51] show that these DNA nanoparticles can effectively transfect both mitotic and terminally differentiated retinal cells. Furthermore, our ability to drive long-term expression (up to four months) suggests that compacted DNA nanoparticles may not be subject to some of the practical impediments that have limited the utility of other forms of non-viral gene therapy.

We have shown herein at least partial structural, functional, and biochemical rescue of the clinically relevant rds^{+/-} RP disease phenotype by delivery of compacted DNA nanoparticles containing wild-type Rds.

The present invention is therefore a method of using compacted nucleic acid nanoparticles for non-viral transfer of the nucleic acids contained therein to various ocular cells, tissues, regions, or sites for the treatment of ocular conditions or diseases.

In one embodiment, the ocular condition or disease may be caused by a genetic defect. Examples of such ocular diseases for which a gene has been identified include, but are not limited to, autosomal retinitis pigmentosa, autosomal dominant retinitis punctata albescens, butterfly-shaped pigment dystrophy of the fovea, adult vitelliform macular dystrophy, Norrie's disease, blue cone monochromasy, choroideremia and gyrate atrophy. These may also be referred to as genetic ocular diseases.

In other embodiments, the ocular disease may not be caused by a specific known genotype (although they may be shown in the future to have a genetic component). These ocular diseases include, but are not limited to, age-related macular degeneration, retinoblastoma, anterior and posterior uveitis, retinovascular diseases, cataracts, inherited corneal defects such as corneal dystrophies, retinal detachment and degeneration and atrophy of the iris, and retinal diseases which are secondary to glaucoma and diabetes, such as diabetic retinopathy.

Ocular diseases which may be treated by the present methods include conditions which are not genetically based but still cause ocular disorders or disfunctions. These include, but are not limited to, viral infections such as Herpes Simplex Virus or cytomegalovirus (CMV) infections, allergic conjunctivitis and other ocular allergic responses, dry eye, lysosomal storage diseases, glycogen storage diseases, disorders of collagen, disorders of glycosaminoglycans and proteoglycans, sphinogolipodoses, mucolipidoses, disorders of amino acid metabolism, dysthyroid eye diseases, anterior and posterior corneal dystrophies, retinal photoreceptor disorders, corneal ulceration and other ocular wounds such as those following surgery.

In a preferred embodiment, the nucleic acid encodes a protein which is expressed, preferably constitutively expressed. In some embodiments, the expression of the exogeneous nucleic acid supplied in the nanoparticle is transient; that is, the exogeneous protein is expressed for a limited time. In other embodiments, the expression is permanent. Thus for example, transient expression systems may be used when therapeutic proteins are to be produced for a short period; for example, certain exogeneous proteins are desirable after ocular surgery or wounding. Alternatively, for on-going or congenital conditions such as retinitis pigmentosa, macular degeneration, or glaucoma, permanent expression may be desired.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In one embodiment, the exogeneous nucleic acid is delivered to corneal epithelial cells. Corneal epithelial cells are subject to injury, allergic reactions and infections, among others. Thus proteins which are useful in the treatment of these conditions, and others, may be delivered via the present invention.

In another embodiment, the exogeneous nucleic acid is delivered to corneal endothelial cells. This is particularly significant since dysfunction of the corneal endothelial cells causes blindness. This layer is often damaged during cataract extraction, which is currently the most common surgical operation in the U.S. In addition, since the corneal endothelium cannot regenerate, since cell division does not occur, the expression of proteins which cause division or regeneration of corneal endothelial cells could be a significant treatment of corneal endothelial damage.

In another embodiment, exogeneous nucleic acid is introduced into the cells of the trabecular meshwork, beneath the periphery of the cornea. The trabecular meshwork is the outflow tract from the anterior chamber of the eye, which allows aqueous humor (the fluid contained within the eye) to drain from the eye. This is significant since glaucoma is a common cause of visual loss in the U.S., and is a result of increased intraocular pressure. Therefore, the methods of the present invention may be useful to regulate the outflow of aqueous humor and treat or cure glaucoma.

In one embodiment, the exogeneous nucleic acid is introduced to cells of the choroid layer of the eye. The choroid layer of the eye is part of the blood supply to the retina, and thus may supply proteins to the retina. For example, BDNF (brain-derived neurotrophic factor) may be delivered in this manner to treat retinal degeneration.

In alternative embodiments, the exogeneous nucleic acid is introduced to cells of the retina, sclera or ciliary body. This last may be done, for example, for controlling production of aqueous fluid in the treatment or prevention of glaucoma.

Similarly, additional embodiments utilize the introduction of exogeneous nucleic acid of the present nanoparticles to the cells of the retinal or ocular vasculature, cells of the vitreous body or cells of the lens, for example the lens epithelium.

As noted above, the nucleic acids of the nanoparticles of the present invention preferably include appropriate sequences that are operably linked to the nucleic acid sequences encoding the protein or RNA to promote its expression in a host cell. “Operably linked” sequences present include both expression control sequences (e.g. promoters) that are contiguous with the coding sequences for the product of interest and expression control sequences that act in trans or at a distance to control the expression of the protein or RNA.

Expression control sequences may include appropriate transcription initiation, termination, promoter and enhancer sequences; efficient RNA processing signals such as splicing and polyadenylation signals; sequences that stabilize cytoplasmic MRNA; sequences that enhance translation efficiency (i.e., Kozak consensus sequence); sequences that enhance protein stability; and when desired, sequences that enhance protein processing and/or secretion. A great number of expression control sequences, e.g., native, constitutive, inducible and/or tissue-specific, are known in the art and may be utilized to drive expression of the gene, depending upon the type of expression desired.

For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence.

The regulatory sequences useful in the constructs of the present invention may also contain an intron, desirably located between the promoter/enhancer sequence and the gene. One possible intron sequence is also derived from SV-40, and is referred to as the SV-40 T intron sequence. Another suitable sequence includes the woodchuck hepatitis virus post-transcriptional element.

The promoter used herein may be made from among a wide number of constitutive or inducible promoters that can express the selected gene or nucleic acid in an ocular cell. In a preferred embodiment, the promoter is cell-specific. The term “cell-specific” means that the particular promoter selected for the recombinant vector can direct expression of the selected gene is a particular ocular cell type. As one example, the promoter is specific for expression of the gene in RPE cells. As another example, the promoter is specific for expression of the gene in photoreceptor cells.

Examples of constitutive promoters which may be included in the nanoparticles of the present invention include, but are not limited to, the RSV LTR promoter/enhancer, the SV40 promoter, the CMV promoter, the dihydrofolate reductase promoter, the phosphoglycerol kinase (PGK) promoter and others previously mentioned or described.

Examples of RPE-specific promoters include, the RPE-65 promoter, the tissue inhibitor of metalloproteinase 3 (Timp3) promoter, the tyrosinase promoter, and the promoters described in International Patent Publication No. WO 00/15822.

Examples of photoreceptor specific promoters include, but are not limited to, the rod opsin promoter, the red-green opsin promoter, the blue opsin promoter, the inter photoreceptor binding protein (IRBP) promoter and the cGMPβ phosphodiesterase promoter, and the promoters described in International Patent Publication No. WO 98/48097. Other promoters which may be used are described in U.S. Pat. Nos. 5,856,152 and 5,871,982.

Alternatively, an inducible promoter may be used to express the gene product, so as to control the amount and timing of the ocular cell's production thereof. Such promoters can be useful if the gene product proves to be toxic to the cell upon excessive accumulation. Inducible promoters include those known in the art and those discussed above including, without limitation, the zinc-inducible sheep metallothionine (MT) promoter; the dexamethasone (Dex)-inducible mouse mammary tumor virus (MMTV) promoter; the T7 promoter; the ecdysone insect promoter; the tetracycline-repressible system; the tetracycline-inducible system; the RU486-inducible system; and the rapamycin-inducible system. Any other type of inducible promoter which is tightly regulated and is specific for the particular target ocular cell type may be used.

Suitability of a particular expression control sequence for a specific gene may be determined by assay and used to choose the expression control sequence which is most appropriate for expression of the desired gene. For example, a target cell may be infected in vitro, and the number of copies of the gene in the cell may be determined by Southern blotting or quantitative polymerase chain reaction (PCR). The level of RNA expression may be determined by Northern blotting or quantitative reverse transcriptase (RT)-PCR; and the level of protein expression may be determined by Western blotting, immunohistochemistry, enzyme-linked immunosorbent assay (ELISA), radioimmunoassay (RIA) or by the specific methods detailed below in the examples.

Ocular-specific genes or nucleic acids contemplated for use in the nanoparticles of the present invention particularly include, but are not limited to: genes encoding opsin protein of rhodopsin (RHO), cyclic GMP phosophodiesterase a-subunit (PDE6A) or β-subunit (PDE6B), the alpha subunit of the rod cyclic nucleotide gated channel (CNGA1), RPE65, RLBP1, ABCR, ABCA4, CRB1, LRAT, CRX, IP1, EFEMP1, peripherin/RDS (PRPH2), ROM1, and arrestin (SAG), which are all known to be mutated in RP, alpha-transducin (GNAT1), rhodopsin kinase (RHOK), guanylate cyclase activator 1A (GUCA1A), retina specific guanylate cyclase (GUCY2D), the alpha subunit of the cone cyclic nucleotide gated cation channel (CNGA3), and cone opsins such as BCP, GCP, and RCP, which are mutated in certain forms of color blindness. Other genes may also be used including those encoding ciliary neurotrophic factor (CNTF), brain derived neurotrophic factor (BDNF), human complement factor H (HCFH), ORF15 variant of Retinitis Pigmentosa GTPase Regulator (RPGR).

Examples of genes which have mutations related to or involved in macular degeneration include CFH (Complement Factor H), CFB (Complement Factor B), ABCR and ACBA4, C2 (Complement Component 2), C3 (Complement Component 3), HTRA1, T2-TrpRS, and RdCVF, and each or all may be used in the nanoparticles and methods contemplated herein to treat, mitigate or prevent macular degeneration conditions.

Other genes specific for ocular conditions or non-specific for ocular conditions, and can be used to treat many forms of ocular conditions. Examples of genes which may be used in their normal form in the nanoparticles of the present invention to treat retinal diseases include (obtained from www.sph.uth.tmc.edu/Retnet/disease.htm), for example: LCA9, NPHP4, RP32, RPE65, ABCA4, COL11A1, GNAT2, PRPF3, SEMA4A, CORD8, HMCN1, AXPC1, CFH, CRB1, RD3, USH2A, RP28, EFEMP1, ALMS1, RP33, CNGA3, MERTK, NPHP1, BBS5, CERKL, KCNJ13, SAG, USH2B, CRV, GNAT1, ATXN7, ARL6, IQCB1, NPHP3, RHO, CLRN1, OPA1, STGD4, MCDR2, PDE6B, WFS1, CC2D2A, PROM1, CNGA1, WFS2, MTP, BBS7, BBS12, RP29, LRAT, CYP4V2, MCDR3, VCAN, GPR98, BSMD, PDE6A, GRM6, C2, CFB, TULP1, MDDC, BBS9, RP9, PEX1, IMPDH1, OPN1SW, CORD9, RP1, TTPA, OPA6, PXMP3, CNGB3, VMD1, KCNV2, TOPORS, INVS, DFNB31, TLR4, TRIM32, RP8, JBTS1, PHYH, ERCC6, RNANC, PCDH15, USH1H, CDH23, RGR, RBP4, PAX2, HTRA1, ARMS2, OAT, TEAD1, USH1C, EVR3, CORS2, ROM1, BEST1, BBS1, VRN1, CABP4, LRP5, MYO7A, FZD4, C1QTNF5, MFRP, CACNA2D4, COL2A1, CODA1, RDH5, BBS10, CEP290, RB1, GRK1, STGD2, ACHM1, MCDR4, NRL, RPGRIP1, LCA3, RDH12, USH1A, TTC8, FBLN5, NR2E3, BBS4, RLBP1, ABCC6, RP22, BBS2, RPGRIP1L, CNGB1, CDH3, FHASD, CACD, GUCY2D, RCD2, AIPL1, PITPNM3, PRPF8, CORD4, UNC119, CA4, USH1G, RGS9, PRCD, FSCN2, OPA4, CORD1, C3, RAX2, RGS9BP, CRX, OPA3, PRPF31, JAG1, MKKS, PANK2, USH1E, OPA5, TIMP3, RP23, RS1, RP6, DMD, A1ED, OPA2, NYX, RPGR, PRD, NPD, CAC, NA1F, RP2, PGK1, CHM, TIMM8A, RP24, COD2, RP34, OPN1LW, OPN1MW, KSS, LHON, MT-TL1, MT-ATP6, MT-TH, and MT-TS2.

These genes, as well as other genes useful for delivery to the eye may be obtained from conventional sources, e.g., from university laboratories or depositories, or synthesized from information obtained from Genbank by techniques well known to persons of ordinary skill in the art.

In a particular embodiment of the method, where the ocular disorder is caused by a mutation in a normal photoreceptor-specific gene, the ocular cells which are the target of the treatment method are the photoreceptor cells. The specific gene which is mutated or absent in the disorder may be the photoreceptor-specific homeo box gene (CRX). Alternatively, the specific gene which is mutated or absent in the disorder may be the retinal guanylate cyclase gene (GUCY2D). In still another embodiment, the gene is a nucleotide sequence encoding RPGR Interacting Protein 1 (RPGRIP1).

Among the ocular disorders, conditions, and diseases that can be treated using the methods of the present invention are severe visual impairment (i.e., blindness), including diseases related to degeneration of cells of the retina and macula, including, but not limited to, Usher syndrome, Stargardt disease, Bardet-Biedl syndrome, Best disease, choroideremia, gyrate-atrophy, retinitis pigmentosa, macular degeneration, Leber Congenital Amaurosis (Leber's Hereditary Optic Neuropathy), Blue-cone monochromacy, retinoschisis, Malattia Leventinese, Oguchi Disease, or Refsum disease, or other diseases related to impairment of the function of the retina or macula.

Other macular degeneration disorders may include but are not limited to any of a number of conditions in which the retinal macula degenerates or becomes dysfunctional, e.g., as a consequence of decreased growth of cells of the macula, increased death or rearrangement of the cells of the macula (e.g., RPE cells), loss of normal biological function, or a combination of these events such as North Carolina macular dystrophy, Sorsby's fundus dystrophy, pattern dystrophy, dominant drusen, and any condition which alters or damages the integrity or function of the macula (e.g., damage to the RPE or Bruch's membrane). For example, the term macular degeneration encompasses retinal detachment, chorioretinal degenerations, retinal degenerations, photoreceptor degenerations, RPE degenerations, mucopolysaccharidoses, rod-cone dystrophies, cone-rod dystrophies and cone degenerations.

Furthermore, the methods disclosed herein for delivering nucleic acids to the eye via non-viral nanoparticles may be used to treat or prevent ocular diseases or conditions, such as the following: maculopathies/retinal degeneration including macular degeneration, including age related macular degeneration (AMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema; Uveitis/retinitis/choroiditis including acute multifocal placoid pigment epitheliopathy, Behcet's disease, birdshot retinochoroidopathy, infectious (syphilis, lyme, tuberculosis, toxoplasmosis) uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome; Vascular diseases/exudative diseases including retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, frosted branch angitis, sickle cell retinopathy and other hemoglobinopathies, angioid streaks, familial exudative vitreoretinopathy, and Eales disease; Traumatic/surgical conditions including sympathetic ophthalmia, uveitic retinal disease, retinal detachment, trauma, laser, PDT, photocoagulation, hypoperfusion during surgery, radiation retinopathy, and bone marrow transplant retinopathy; Proliferative disorders including proliferative vitreal retinopathy and epiretinal membranes, and proliferative diabetic retinopathy; Infectious disorders including ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, bacterial diseases, ocular syphilis, ocular tuberculosis, diffuse unilateral subacute neuroretinitis, and myiasis; Genetic disorders including retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; Retinal tears/holes including retinal detachment, macular hole, and giant retinal tear; Tumors including retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors and miscellaneous conditions including punctate inner choroidopathy, acute posterior multifocal placoid pigment epitheliopathy, myopic retinal degeneration, and acute retinal pigment epithelitis.

It is contemplated that these and other disorders and diseases will be treated by delivery of via non-viral nanoparticles of genes, oligonucleotides, expression plasmids, siRNA and shRNA (for examples of such siRNAs and shRNAs which may be used herein see for example U.S. Pat. No. 7,176,304), linear, circular, and supercoiled plasmid DNA or other forms of nucleic acids to the affected tissues which, due to their impairment or defectiveness are responsible for the disorders or disease conditions.

In a preferred embodiment, the invention is directed to the use of nanoparticles comprising the normal versions of one or more of the genes CA4, CRX, FSCN2, GUCA1B, IMPDH1, NR2E3, NRL, PRPF3, PRPF8, PRPF31, PRPH2, RHO, ROM1, RP1, RP9, SEMA4A, TOPORS, ABCA4, CERKL, CNGA1, CNGB1, CRB1, LRAT, MERTK, NRL, PDE6A, PDE6B, PRCD, PROM1, RGR, RLBP1, RP1, RPE65, SAG, TULP1, USH2A, RP2, and RPGR for use in treating autosomal dominant, autosomal recessive, or X-linked forms of retinitis pigmentosa.

In another preferred embodiment, the invention is directed to the use of nanoparticles comprising the normal versions of one or more of genes ABCA4, ARMS2, C2, C3, CFB, CFH, ERCC6, FBLN5, HMCN1, HTRA1, RAX2 and TLR4 for use in treating age-related macular degeneration (AMD) and one or more of genes BEST1, C1QTNF5, EFEMP1, ELOVL4, FSCN2, GUCA1B, PRPH2, TIMP3, and RPGR for use in treating autosomal dominant macular degeneration, autosomal recessive macular degeneration, or X-linked macular degeneration.

The method of treating a patient may comprise administering the compacted nanoparticles to the patient by at least one of intravitreal placement, subretinal placement, subconjuctival placement, conjunctival placement, anterior chamber placement, episcleral placement, sub-tenon placement, retrobulbar placement, suprachoroidal placement, and systemic injection via intravenous and/or intraarterial administration. Placement methods may include injection and/or surgical insertion. Preferably the compacted nanoparticle is administered via intravitreal injection or subretinal injection. In a preferred embodiment of the invention the amount of nucleic acid per dosage is provided to the subjects eye at a concentration of 0.01 μg/μl to 10 μg/μl, depending on the desired level of expression in the ocular cells. Individual dosages may range (in non-limiting examples) for example from 1 μl to 1000 μl, and more preferably are from 10 μl to 100 μl. The nanoparticles may be provided in a composition comprising any pharmaceutically-aceptable carrier, such as a saline solution (e.g., PBS).

As indicated elsewhere herein, the present invention in a preferred embodiment is a method of treating a subject having an ocular disorder, comprising providing a compacted nanoparticle having a minor diameter below 25 nm and which comprises a nucleic acid covalently linked to a cationic polymeric material; and administering the compacted nanoparticle to a tissue of the eye of the patient for treating the ocular disorder. In preferred embodiments, the ocular condition or disorder to be treated is related to retinal and macular degeneration, Usher syndrome, Stargardt disease, Bardet-Biedl syndrome, Best disease, choroideremia, gyrate-atrophy, retinitis pigmentosa, Leber Congenital Amaurosis (Leber's Hereditary Optic Neuropathy), various types of optic neuropathy and optic neuritis, Blue-cone monochromacy, retinoschisis, Malattia Leventinese, Oguchi Disease, and Refsum disease, retinal detachment, chorioretinal degenerations, retinal degenerations, photoreceptor degenerations, degeneration of the retinal pigment epithelium, mucopolysaccharidoses, rod-cone dystrophies, cone-rod dystrophies, cone degenerations, conditions involving decreased growth of cells of the macula, increased death or rearrangement of the retinal pigment epithelial cells of the macula, North Carolina macular dystrophy, Sorsby's fundus dystrophy, pattern dystrophy, dominant drusen, or any condition which alters or damages the integrity or function of the macula.

The method of the present invention may use any gene described herein but, in particular embodiments, nanoparticles comprising a nucleic acid or gene or cDNA which encodes at least one of opsin protein of rhodopsin (RHO), cyclic GMP phosophodiesterase α-subunit (PDE6A) or, β-subunit (PDE6B), the alpha subunit of the rod cyclic nucleotide gated channel (CNGA1), RPE65, RLBP1, ABCR, ABCA4, CRB1, LRAT, CRX, IP1, EFEMP1, peripherin/RDS, ROM1, arrestin (SAG), alpha-transducin (GNAT1), rhodopsin kinase (RHOK), guanylate cyclase activator 1A (GUCA1A), retina specific guanylate cyclase (GUCY2D), the alpha subunit of the cone cyclic nucleotide gated cation channel (CNGA3), and cone opsins BCP, GCP, and RCP. Other genes may also be used including those encoding ciliary neurotrophic factor (CNTF), brain derived neurotrophic factor (BDNF), and ORF15 variant of Retinitis Pigmentosa GTPase Regulator (RPGR). Examples of genes which are related to macular degeneration include CFH (Complement Factor H), CFB (Complement Factor B), ABCR and ACBA4, C2 (Complement Component 2), C3 (Complement Component 3), HTRA1, T2-TrpRS, and RdCVF and may be used in the nanoparticles and methods contemplated herein to treat, mitigate or prevent macular degeneration conditions.

Other genes specific for ocular conditions or non-specific for ocular conditions and can be used to treat many forms of ocular conditions. In the method of the present invention the ocular cells or tissues which are treated may be selected from the group consisting of cells located in the ganglion cell layer (GCL), the inner plexiform layer (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the cornam, and epithelium of ocular sebaceous glands. Genes which cause retinal diseases in these cells and tissues and which may be used in the nanoparticles used in the present methods are described elsewhere herein.

In one preferred embodiment, the nanoparticles used in the present invention comprise DNA and CK30PEG10k (a 30-mer lysine polycationic peptide having an N-terminal cysteine which is conjugated via a covalent linkage to 10 kDa polyethylene glycol) and have rod or ellipsoid shapes (depending on whether acetate or trifluoroacetate is used as the lysine counterion (respectively) during compaction) and have minor diameters of less than 25 nm. Other polycation and counterion molecules which may be used in the present invention are discussed above or are shown in U.S. Published Patent Applications and Patents previously cited herein.

All articles, publications, patents and published patent applications indicated herein are hereby expressly incorporated herein by reference in their entireties.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, compositions of matter, means, methods and steps described in the specification. As one of ordinary skill in the art will readily appreciate from the disclosure of the present invention, processes, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, compositions of matter, means, methods, or steps.

### REFERENCES

1. Berry V, Francis P, Kaushal S, Moore A, Bhattacharya S (2000) Missense mutations in MIP underlie autosomal dominant ‘polymorphic’ and lamellar cataracts linked to 12q. Nat Genet 25: 15-17.
2. Stone E M, Fingert J H, Alward W L, Nguyen T D, Polansky J R, et al. (1997) Identification of a gene that causes primary open angle glaucoma. Science 275: 668-670.
3. Farrar G J, Kenna P F, Humphries P (2002) On the genetics of retinitis pigmentosa and on mutation-independent approaches to therapeutic intervention. Embo J 21:857-864.
4. Ferreira P A (2005) Insights into X-linked retinitis pigmentosa type 3, allied diseases and underlying pathomechanisms. Hum Mol Genet 14 Suppl 2: R259-267.
5. Molday R S (1998) Photoreceptor membrane proteins, phototransduction, and retinal degenerative diseases. The Friedenwald Lecture. Invest Opthalmol Vis Sci 39: 2491-2513.
6. Bennett J (2006) Commentary: an aye for eye gene therapy. Hum Gene Ther 17: 177-179.
7. Batten M L, Imanishi Y, Tu D C, Doan T, Zhu L, et al. (2005) Pharmacological and rAAV gene therapy rescue of visual functions in a blind mouse model of Leber congenital amaurosis. PLOS Med 2: e333.
8. Drenser K A, Timmers A M, Hauswirth W W, Lewin A S (1998) Ribozyme-targeted destruction of RNA associated with autosomal-dominant retinitis pigmentosa. Invest Opthalmol Vis Sci 39: 681-689.
9. Lewin A S, Drenser K A, Hauswirth W W, Nishikawa S, Yasumura D, et al. (1998) Ribozyme rescue of photoreceptor cells in a transgenic rat model of autosomal dominant retinitis pigmentosa. Nat Med 4: 967-971.
10. Sarra G M, Stephens C, de Alwis M, Bainbridge J W, Smith A J, et al. (2001) Gene replacement therapy in the retinal degeneration slow (rds) mouse: the effect on retinal degeneration following partial transduction of the retina. Hum Mol Genet 10: 2353-2361.
11. Rolling F (2004) Recombinant AAV-mediated gene transfer to the retina: gene therapy perspectives. Gene Ther 11 Suppl 1: 526-32.
12. Bennett J, Tanabe T, Sun D, Zeng Y, Kjeldbye H, et al. (1996) Photoreceptor cell rescue in retinal degeneration (rd) mice by in vivo gene therapy. Nat Med 2: 649-654.
13. Chen Y, Moiseyev G, Takahashi Y, Ma J X (2006) RPE65 gene delivery restores isomerohydrolase activity and prevents early cone loss in Rpe65−/− mice. Invest Opthalmol Vis Sci 47: 1177-1184.
14. Ali R R, Sarra G M, Stephens C, Alwis M d., Bainbridge J W, et al. (2000) Restoration of photoreceptor ultrastructure and function in retinal degeneration slow mice by gene therapy. Nat Genet 25: 306-310.
15. Dinculescu A, Glushakova L, Min S H, Hauswirth W W (2005) Adeno-associated virus-vectored gene therapy for retinal disease. Hum Gene Ther 16: 649-663.
16. Aguirre G D, Baldwin V, Pearce-Kelling S, Narfstrom K, Ray K, et al. (1998) Congenital stationary night blindness in the dog: common mutation in the RPE65 gene indicates founder effect. Mol Vis 4:23.
17. Marlhens F, Bareil C, Griffoin J M, Zrenner E, Amalric P, et al. (1997) Mutations in RPE65 cause Leber's congenital amaurosis. Nat Genet 17: 139-141.
18. Hong D H, Pawlyk B S, Adamian M, Sandberg M A, Li T (2005) A single, abbreviated RPGR-ORF15 variant reconstitutes RPGR function in vivo. Invest Opthalmol Vis Sci 46: 435-441.
19. Thomas C E, Ehrhardt A, Kay Mass. (2003) Progress and problems with the use of viral vectors for gene therapy. Nat Rev Genet 4: 346-358.
20. Halbert C L, Miller A D, McNamara S, Emerson J, Gibson R L, et al. (2006) Prevalence of neutralizing antibodies against adeno-associated virus (AAV) types 2, 5, and 6 in cystic fibrosis and normal populations: Implications for gene therapy using AAV vectors. Hum Gene Ther 17: 440-447.
21. Ott M G, Schmidt M, Schwarzwaelder K, Stein S, Siler U, et al. (2006) Correction of X-linked chronic granulomatous disease by gene therapy, augmented by insertional activation of MDS1-EVI1, PRDM16 or SETBP1. Nat Med 12: 401-409.
22. Baum C, Kustikova O, Modlich U, Li Z, Fehse B (2006) Mutagenesis and oncogenesis by chromosomal insertion of gene transfer vectors. Hum Gene Ther 17: 253-263.
23. Raper S E, Chirmule N, Lee F S, Wivel N A, Bagg A, et al. (2003) Fatal systemic inflammatory response syndrome in a ornithine transcarbamylase deficient patient following adenoviral gene transfer. Mol Genet Metab 80: 148-158.
24. Liu G, Li D, Pasumarthy M K, Kowalczyk T H, Gedeon C R, et al. (2003) Nanoparticles of compacted DNA transfect postmitotic cells. J Biol Chem 278: 32578-32586.
25. Ziady A G, Gedeon C R, Muhammad O, Stillwell V, Oette S M, et al. (2003) Minimal toxicity of stabilized compacted DNA nanoparticles in the murine lung. Mol Ther 8: 948-956.
26. Ziady A G, Gedeon C R, Miller T, Quan W, Payne J M, et al. (2003) Transfection of airway epithelium by stable PEGylated poly-L-lysine DNA nanoparticles in vivo. Mol Ther 8: 936-947.
27. Fink T L, Klepcyk P J, Oette S M, Gedeon C R, Hyatt S L, et al. (2006) Plasmid size up to 20 kbp does not limit effective in vivo lung gene transfer using compacted DNA nanoparticles. Gene Ther 13:1048-1051.
28. Kowalczyk T H, Pasumarthy, M. K., Gedeon, C., Hyatt, S., Payne, J. M., Muhammad, O., Moen, R. C., and Cooper, M. J. (2001) Type of polylysine counterion influences morphology and biological function of condensed DNA. Mol Ther 3: S359.
29. Konstan M W, Davis P B, Wagener J S, Hilliard K A, Stern R C, et al. (2004) Compacted DNA nanoparticles administered to the nasal mucosa of cystic fibrosis subjects are safe and demonstrate partial to complete cystic fibrosis transmembrane regulator reconstitution. Hum Gene Ther 15: 1255-1269.
30. Mohan R R, Sharma A, Netto M V, Sinha S, Wilson S E (2005) Gene therapy in the cornea. Prog Retin Eye Res 24: 537-559.
31. Jun A S, Larkin D F (2003) Prospects for gene therapy in corneal disease. Eye 17: 906-911.
32. Martin K R, Quigley H A (2004) Gene therapy for optic nerve disease. Eye 18: 1049-1055.
33. Atencio I A, Chen Z, Nguyen Q H, Faha B, Maneval D C (2004) p21WAF-1/Cip-1 gene therapy as an adjunct to glaucoma filtration surgery. Curr Opin Mol Ther 6: 624-628.
34. Martin K R, Quigley H A, Zack D J, Levkovitch-Verbin H, Kielczewski J, et al. (2003) Gene therapy with brain-derived neurotrophic factor as a protection: retinal ganglion cells in a rat glaucoma model. Invest Opthalmol Vis Sci 44: 4357-4365.
35. Yin Y, Henzl M T, Lorber B, Nakazawa T, Thomas T T, et al. (2006) Oncomodulin is a macrophage-derived signal for axon regeneration in retinal ganglion cells. Nat Neurosci 9:843-852.
36. Bi A, Cui J, Ma Y P, Olshevskaya E, Pu M, et al. (2006) Ectopic expression of a microbial-type rhodopsin restores visual responses in mice with photoreceptor degeneration. Neuron 50: 23-33.
37. Peeters L, Sanders N N, Braeckmans K, Boussery K, Van de Voorde J, et al. (2005) Vitreous: a barrier to nonviral ocular gene therapy. Invest Opthalmol Vis Sci 46: 3553-3561.
38. Cashman S M, Binkley E A, Kumar-Singh R (2005) Towards mutation-independent silencing of genes involved in retinal degeneration by RNA interference. Gene Ther 12: 1223-1228.
39. Gariano R F, Gardner T W (2005) Retinal angiogenesis in development and disease. Nature 438: 960-966.
40. G. M. Acland, G. D. Aguirre, J. Ray et al., Nature genetics 28 (1), 92 (2001).
41. R. R. Ali, G. M. Sarra, C. Stephens et al., Nature genetics 25 (3), 306 (2000).
42. D. Luo and W. M. Saltzman, Nature biotechnology 18 (8), 893 (2000).
43. X. Cai, S. Conley, and M. Naash, Vision research 48 (3), 319 (2008).
44. M J Cooper, Molecular therapy S15, Vii Scientific symposium 315: Non (2007).
45. P. B. Davis and M. J. Cooper, The AAPS journal 9 (1), E11 (2007).
46. T. L. Fink, P. J. Klepcyk, S. M. Oette et al., Gene Ther 13 (13), 1048 (2006).
47. G. Liu, D. Li, M. K. Pasumarthy et al., The Journal of biological chemistry 278 (35), 32578 (2003).
48. D. Luo and W. M. Saltzman, Nature biotechnology 18 (1), 33 (2000).
9. D. M. Yurek, A. Fletcher-Turner, and M. J. Cooper, Mol Ther 11, 253 (2005).
50. X. Chen, D. M. Kube, M. J. Cooper et al., Mol Ther 16 (2), 333 (2008).
51. R. Farjo, J. Skaggs, A. B. Quiambao et al., PLoS ONE 1, e38 (2006).
52. M. W. Konstan, P. B. Davis, J. S. Wagener et al., Human gene therapy 15 (12), 1255 (2004).
53. A. G. Ziady, C. R. Gedeon, T. Miller et al., Mol Ther 8 (6), 936 (2003).
54. G. Connell, R. Bascom, L. Molday et al., Proceedings of the National Academy of Sciences of the United States of America 88 (3), 723 (1991).
55. G. J. Connell and R. S. Molday, Biochemistry 29 (19), 4691 (1990).
56. R. S. Molday, D. Hicks, and L. Molday, Investigative opthalmology & visual science 28 (1), 50 (1987).
57. G. J. Farrar, P. Kenna, S. A. Jordan et al., Nature 354 (6353), 478 (1991).
58. F. J. Khoubian, E. P. Shakin, A. Tantri et al., Retina (Philadelphia, Pa. 25 (8), 999 (2005).
59. C. Portera-Cailliau, C. H. Sung, J. Nathans et al., Proceedings of the National Academy of Sciences of the United States of America 91 (3), 974 (1994).
60. B. L. Lam, K. Vandenburgh, V. C. Sheffield et al., American journal of opthalmology 119 (1), 65 (1995).
61. M. Nour, X. Q. Ding, H. Stricker et al., Investigative opthalmology & visual science 45 (8), 2514 (2004).
62. R. K. Hawkins, H. G. Jansen, and S. Sanyal, Exp Eye Res 41 (6), 701 (1985).
63. T. Cheng, N. S. Peachey, S. Li et al., J Neurosci 17 (21), 8118 (1997).
64. J. Ma, J. C. Norton, A. C. Allen et al., Genomics 28 (2), 212 (1995).
65. W. Kedzierski, D. Bok, and G. H. Travis, Journal of neurochemistry 72 (1), 430 (1999).
66. G. M. Sarra, C. Stephens, M. de Alwis et al., Hum Mol Genet 10 (21), 2353 (2001).
67. F. C. Schlichtenbrede, L. da Cruz, C. Stephens et al., The journal of gene medicine 5 (9), 757 (2003).
68. E. S. Lee, B. Burnside, and J. G. Flannery, Investigative opthalmology & visual science 47 (5), 2150 (2006).
69. G. I. Liou, L. Geng, M. R. al-Ubaidi et al., The Journal of biological chemistry 265 (15), 8373 (1990).
70. C. L. Cepko, C. P. Austin, X. Yang et al., Proceedings of the National Academy of Sciences of the United States of America 93 (2), 589 (1996).
71. J. Lem, N. V. Krasnoperova, P. D. Calvert et al., Proceedings of the National Academy of Sciences of the United States of America 96 (2), 736 (1999).
72. L. D. Carter-Dawson and M. M. LaVail, J Comp Neurol 188 (2), 245 (1979).
73. L. D. Carter-Dawson and M. M. LaVail, J Comp Neurol 188 (2), 263 (1979).
74. C. Li, M. Cheng, H. Yang et al., Optom Vis Sci 78 (6), 425 (2001).
75. M. Nour, A. B. Quiambao, W. M. Peterson et al., Investigative opthalmology & visual science 44 (10), 4505 (2003).
76. T. Matsuda and C. L. Cepko, Proceedings of the National Academy of Sciences of the United States of America 101 (1), 16 (2004).
77. R. Farjo, J. S. Skaggs, B. A. Nagel et al., The Journal of cell biology 173 (1), 59 (2006).
78. M. W. Pfaffl, Nucleic Acids Res 29 (9), e45 (2001).
79. X. Q. Ding, M. Nour, L. M. Ritter et al., Hum Mol Genet 13 (18), 2075 (2004).
80. C. Li, X. Q. Ding, J. O'Brien et al., Investigative opthalmology & visual science 44 (6), 2433 (2003).
81. E. Tan, Q. Wang, A. B. Quiambao et al., Investigative opthalmology & visual science 42 (3), 589 (2001).
82. Rosenfeld P J, Heier J S, Hantsbarger G, Shams N (2006) Tolerability and efficacy of multiple escalating doses of ranibizumab (Lucentis) for neovascular age-related macular degeneration. Opthalmology 113:632.
83. Ng E W, Shima D T, Calias P, Cunningham E T, Jr., Guyer DR, et al. (2006) Pegaptanib, a targeted anti-VEGF aptamer for ocular vascular disease. Nat Rev Drug Discov 5: 123-132.

What is claimed is:

1. A method of treating a subject having an ocular disorder, comprising:
providing a non-viral nanoparticle comprising a compacted nucleic acid molecule linked to a polymer component, wherein the nucleic acid molecule is DNA or RNA and wherein the non-viral nanoparticle has a minor diameter which is less than 25 nm; and
administering the non-viral nanoparticle to an eye of the subject in a manner such that the nucleic acid molecule is transfected within an ocular cell of the eye of the subject and is able to be expressed therein.

2. The method of claim 1 wherein the nucleic acid molecule comprises a gene sequence and a promoter sequence.

3. The method of claim 2 wherein the nucleic acid molecule is constitutively expressed within the ocular cell.

4. The method of claim 1 wherein the non-viral nanoparticle is administered via injection into the subretinal space.

5. The method of claim 1 wherein the non-viral nanoparticle is administered via intravitreal injection.

6. The method of claim 1 wherein the non-viral nanoparticle has an ellipsoidal shape.

7. The method of claim 1 wherein the non-viral nanoparticle has a rod-like shape.

8. The method of claim 1 wherein the ocular disorder to be treated is a disease of the retina or of a portion thereof.

9. The method of claim 8 wherein the ocular disorder is Usher syndrome, Stargardt disease, Bardet-Biedl syndrome, Best disease, choroideremia, gyrate-atrophy, retinitis pigmentosa, macular degeneration, Leber Congenital Amaurosis (Leber's Hereditary Optic Neuropathy), Blue-cone monochromacy, retinoschisis, Malattia Leventinese, Oguchi Disease, or Refsum disease.

10. The method of claim 1 wherein the nucleic acid molecule comprises at least one of the genes CA4, CRX, FSCN2, GUCA1B, IMPDH1, NR2E3, NRL, PRPF3, PRPF8, PRPF31, PRPH2, RHO, ROM1, RP1, RP9, SEMA4A, TOPORS, ABCA4, CERKL, CNGA1, CNGB1, CRB1, LRAT, MERTK, NRL, PDE6A, PDE6B, PRCD, PROM1, RGR, RLBP1, RP1, RPE65, SAG, TULP1, USH2A, RP2, and RPGR.

11. The method of claim 1 wherein the nucleic acid molecule comprises at least one of the genes ABCA4, ARMS2, C2, C3, CFB, CFH, ERCC6, FBLN5, HMCN1, HTRA1, RAX2 and TLR4, BEST1, C1QTNF5, EFEMP1, ELOVL4, FSCN2, GUCA1B, PRPH2, TIMP3, and RPGR.

12. The method of claim 1 wherein the ocular cell transfected by the non-viral nanoparticle is a cell of the retina, retinal pigment epithelium, macula, ganglion cell layer, inner plexiform layer, inner nuclear layer, outer plexiform layer, outer nuclear layer, outer segments or inner segments of rods and cones, epithelium of the conjunctiva, iris, ciliary body, cornea, or ocular sebaceous gland epithelia.

13. A compacted nanoparticle, comprising:
a nucleic acid linked to a polymer component, the nucleic acid comprising at least one of CA4, CRX, FSCN2, GUCA1B, IMPDH1, NR2E3, NRL, PRPF3, PRPF8, PRPF31, PRPH2, RHO, ROM1, RP1, RP9, SEMA4A, TOPORS, ABCA4, CERKL, CNGA1, CNGB1, CRB1, LRAT, MERTK, NRL, PDE6A, PDE6B, PRCD, PROM1, RGR, RLBP1, RP1, RPE65, SAG, TULP1, USH2A, RP2, RPGR, ABCA4, ARMS2, C2, C3, CFB, CFH, ERCC6, FBLN5, HMCN1, HTRA1, RAX2, TLR4, BEST1, C1QTNF5, EFEMP1, ELOVL4, FSCN2, GUCA1B, PRPH2, TIMP3, and RPGR; and
a promoter sequence; and
wherein the nanoparticle has a minor diameter of less than 25 nm, and wherein the polymer component comprises a PEG molecule covalently linked to a peptide component comprising a terminal cysteine molecule and multiple repeating lysine or arginine residues.

14. A therapeutic nucleic acid composition comprising the nanoparticle of claim 13 disposed within a pharmaceutically-acceptable carrier or vehicle.
